(19) United States
(12) Patent Application Publication
(10) Pub. No.: US 2022/0148688 A1
(43) Pub. Date: May 12, 2022

(54) SYSTEM, PROGRAM, AND METHOD

(71) Applicant: A&T CORPORATION, Fujisawa-shi, Kanagawa (JP)

(72) Inventors: Kentaro NAKAJIMA, Yokohama (JP); Mayu NAGAO, Yokohama (JP)

(21) Appl. No.: 17/283,142

(22) PCT Filed: Jul. 4, 2019
(86) PCT No.: PCT/JP2019/026637

§ 371 Date: Jan. 7, 2022

(30) Foreign Application Priority Data
Oct. 10, 2018 (JP) 2018-192010

(51) Int. Cl. G16H 10/20; G06F 16/27

(52) U.S. Cl. CPC G16H 10/20; G06F 16/27

(57) ABSTRACT

Medical Data Processing System 101 reduces a workload for specifying a suitable reference range that is used for assessing a validity of a result of medical examination. Examination Result Extracting Unit 313 extracts from Examination Result Database 103 datasets indicating examination results that meet a first extraction condition. Examination Result Extracting Unit 313 generates Work Database 104 for temporarily storing the extracted datasets, and causes External Storage Device 205 to store Work Database 104. Reading Unit 314 extracts from Work Database 104 datasets indicating examination results that meet a second extraction condition. Distribution Chart Generating Unit 316 specifies, as a reference range, a range within which examination results indicated by datasets that are not rejected from the datasets extracted from Work Database 104 in accordance with a rejection rate are distributed. Zone Displaying Unit 318 causes Display 206 to display a distribution chart with an x-axis representing a previous result of examination and a y-axis representing a current result of examination performed for the same examinee that indicates the reference range.

### TECHNICAL FIELD

The present invention relates to a system, a program, and a method. In particular, the present invention relates to a system, a program and a method for reducing a burden on an operator by improving an efficiency of processing procedures for an operation of specifying a reference range for confirming a validity of a new result of a clinical examination based on past examination results stored in a database or the like, and for an operation of assessing the specified reference range.

### BACKGROUND ART

To determine a health condition of a patient undergoing treatment or that of a healthy person undergoing a health check, samples of blood and urine are collected, and the samples are subject to clinical examination. Sometimes, an examination result that is likely to be abnormal may be acquired due to, for example, a sample mix-up, an operation error, or the like. Therefore, it is necessary to assess whether a result of a clinical examination is within or outside a reference range, and in a case that the assessment result is outside the reference range, it is necessary to carry out a re-examination.

Prior to carrying out the assessment, it is necessary to determine a reference range for each item included in a clinical examination. An optimum reference range for each item may differ depending on a gender of an examinee, a hospital department in which the examinee was examined, and whether the examinee is an outpatient or an inpatient, etc. As a technique for determining with a high probability whether an examination result shows a normal value or an abnormal value, there is disclosed in Patent Document 1, for example, an automatic analyzer that performs in real time several types of checks, which are respectively referred to as an upper and lower limit value check, a measurement limit value check, an inter-item correlation check, a previous measurement value check for comparison with a previous measurement value, and a combination check to check for sample mix-up using a previous measurement value.

Further, there is disclosed in Patent Document 2, for example, a clinical examination analyzer that sets a reference value for assessing whether an examination-result value is appropriate based on a number of days that has elapsed from a date of a previous examination of the same examinee, or based on the number of days that has elapsed and the examination-result value, and evaluates appropriateness of a value of a new examination result in accordance with the reference value. To determine reference values for assessing examination results, statistical processes are carried out based on past results of the same types of examination stored in a database.

Further, there is disclosed in Patent Document 3, for example, an inspection result display device that displays a distribution chart with a previous-value axis and a current-value axis where there are plotted values of examination results that satisfy predetermined conditions, along with boundary lines showing conditions before and after a change for assessing previous examination-result values and current examination-result values, and whether differences between the previous examination-result values and the current examination-result values, or ratios between the previous examination-result values and the current examination-result values are within an appropriate range for each of a clinical examination item.

### PRIOR ART DOCUMENT

### Patent Document

Patent Document 1: Japanese Unexamined Patent Application No. Sho 61-235753

### Patent Document 2: Japanese Patent No. 2828609

### Patent Document 3: Japanese Patent No. 5297750

### SUMMARY OF THE INVENTION

### Problem to be Solved by the Invention

When a reference range for assessing whether an examination result is likely an abnormal value is assessed based on past examination results that, it is necessary to consider a number of past examination results used for determining the reference range. When the number of past examination results used for determining the reference range is particularly small, the determined reference range is susceptible to an individual examination result, and there is a high possibility of deviation in the determined reference range. If such a deviation occurs in the reference range, appropriateness of the examination result cannot be properly assessed, and an abnormal value that should be reported to a physician may be overlooked, or an unnecessary re-examination may be carried out, etc.

On the other hand, when the number of past examination results used for determining the reference range is particularly large, considerable time is required to determine the reference range. For example, the clinical examination analyzer disclosed in the above-mentioned Patent Document 2 determines an optimum reference range using past examination results extracted using extraction conditions relating to attribute information of examinees such as gender, hospital department, inpatient/outpatient classification, etc. Accordingly, each time the extraction conditions change, it is necessary to re-extract a large number of past examination results used for determining the optimum reference range from past examination results stored in a database, and to process the re-extracted past examination results, which takes a considerable amount of time. In particular, when a plurality of reference ranges are determined and the determined reference ranges are evaluated, a considerable amount of time is required.

When an operator optimizes a reference range, it is desirable for the operator to be able to assess distribution charts of examination results before and after the extraction conditions change. In the following, an area where examination results which are likely normal are distributed in a distribution chart is referred to as a “zone.” To generate and display distribution charts, it is necessary to extract from a database used for storing past examination results a large number of examination results each time extraction conditions change. Accordingly, for the operator to optimize the reference range a considerable amount of time is required since a distribution chart is required to be redrawn each time the extraction conditions change.

It is a burden for the operator to concurrently perform the above-described time-consuming operations between carrying out examination operations for which results are required to be promptly reported to physicians.

Accordingly, it is an object of the present invention to reduce a burden imposed on an operator in determining a reference range for assessing appropriateness of clinical examination results.

### Means for Solving the Problem

To solve the problem described above, the present invention includes, as a first aspect, a system comprising: a first acquiring unit that acquires a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee; a generating unit that extracts from the first database datasets that satisfy the first extraction condition, and generates a second database that stores the datasets extracted from the first database; a storage unit that stores the second database; a second acquiring unit that acquires a second extraction condition dataset indicating a second extraction condition for extracting datasets from the second database, the second extraction condition relating to at least one of an item of extraction, a period of examination, and an attribute of an examinee; a third acquiring unit that acquires a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions; an extraction unit that extracts from the second database datasets that satisfy the second extraction condition; a specifying unit that specifies a reference range within which samples that are not rejected from a population in accordance with the rejection rate are distributed, the population being generated based on the datasets extracted from the second database and consisting of samples, each of which indicates a result of the first examination and a result of the second examination; and a display unit that displays a graph with a first coordinate axis representing a result of the first examination and a second coordinate axis representing a result of the second examination, the graph indicating the reference range.

The present invention includes, as a second aspect, a system according to the first aspect, wherein: when the second acquiring unit acquires a new second extraction condition dataset to be replaced with the second extraction condition dataset already acquired by the second acquiring unit; the extraction unit extracts from the second database datasets that satisfy a new second extraction condition indicated by the new second extraction condition dataset; the specifying unit specifies a new reference range by use of the datasets extracted from the second database by use of the new second extraction condition; and the display unit displays a new graph indicating the new reference range instead of the graph displayed thus far.

The present invention includes, as a third aspect, a system according to the first or second aspect, wherein: when the third acquiring unit acquires a new rejection rate dataset to be replaced with the rejection rate dataset already acquired by the third acquiring unit; the specifying unit specifies a new reference range in accordance with a new rejection rate indicated by the new rejection rate dataset; and the display unit displays a new graph indicating the new reference range instead of the graph displayed thus far.

The present invention includes, as a fourth aspect, a system according to any one of the first to third aspects, wherein: when the first examination is performed at a first timing and the second examination is performed at a second timing later than the first timing; the system further comprises a fourth acquiring unit that acquires a boundary value dataset indicating one or more boundary values of a number of days elapsed between the first timing and the second timing, the one or more boundary values being used for classifying datasets into groups; the specifying unit specifies a reference range for each of groups of the datasets extracted from the second database classified by use of the one or more boundary values; and the display unit displays graphs indicating the reference ranges specified by the specifying unit for each of the groups.

The present invention includes, as a fifth aspect, a system according to the fourth aspect, wherein: when the fourth acquiring unit acquires a new boundary value dataset to be replaced with the boundary value dataset already acquired by the fourth acquiring unit; the specifying unit specifies a new reference range for each of new groups of the datasets extracted from the second database classified by use of one or more boundary values indicated by the new boundary value dataset; and the display unit displays graphs indicating the new reference ranges specified by the specifying unit for each of the new groups.

The present invention includes, as a sixth aspect, a system according to the fourth or fifth aspect, wherein: the display unit displays a first interface screen that includes a graph indicating a number of samples in the population of each number of days elapsed between the first timing and the second timing, the first interface screen receiving an operation carried out by a user on the graph; and the fourth acquiring unit acquires a boundary value dataset indicating one or more boundary values that are input by the user via the first interface screen.

The present invention includes, as a seventh aspect, a system according to any one of the first to sixth aspects, wherein: the system further comprises a fifth acquiring unit that acquires an axis range dataset indicating displayed ranges of a first coordinate axis and a second coordinate axis of a graph indicating a reference range; the display unit displays a graph indicating the reference range specified by the specifying unit, the graph having a first coordinate axis and a second coordinate axis whose displayed ranges are indicated by the axis range dataset; and when the fifth acquiring unit acquires a new axis range dataset to be replaced with the axis range dataset already acquired by the fifth acquiring unit, the display unit displays a new graph indicating the reference range with the first coordinate axis and the second coordinate axis whose displayed ranges are indicated by the new axis range dataset instead of the graph displayed thus far.

The present invention includes, as an eighth aspect, a system according to the seventh aspect, wherein: the display unit displays a second interface screen that includes a graph indicating a number of samples in a population of each examination-result value, the population being generated based on the datasets extracted from the second database, the second interface screen receiving an operation carried out by a user on the graph; and the fifth acquiring unit acquires an axis range dataset indicating a range of the first coordinate axis and the second coordinate axis that is input by the user via the second interface screen.

The present invention includes, as a ninth aspect, a system according to any one of the first to eighth aspects, wherein: the display unit displays a graph including a distribution chart indicating a distribution of samples in a population and the reference range on a same coordinate plane, the population being generated based on the datasets extracted from the second database and consisting of samples each of which indicates a result of the first examination and a result of the second examination.

The present invention includes, as a tenth aspect, a system according to the ninth aspect, wherein: the display unit displays a third interface screen that includes the graph including the distribution chart and the reference range, the third interface screen receiving an operation carried out by a user on the graph; and the display unit displays information on a sample specified by the user via the third interface screen.

The present invention includes, as an eleventh aspect, a system according to any one of the first to tenth aspects, wherein: the system further comprises a sixth acquiring unit that acquires datasets for assessment, the datasets for assessment relating to clinical examinations, each of the datasets for assessment indicating an item of examination, an examination-result value, a date of examination, an identifier of an examinee, and an attribute of the examinee, the datasets for assessment being different from the datasets extracted from the second database by use of the second extraction condition; and the display unit displays a graph including a distribution chart indicating distribution of samples in a population and the reference range on a same coordinate plane, the population being generated based on the datasets for assessment and consisting of samples each of which indicates a result of the first examination and a result of the second examination.

The present invention includes, as a twelfth aspect, a system according to the eleventh aspect, wherein: the extraction unit extracts from the second database the datasets for assessment by use of an extraction condition that is different from the second extraction condition.

The present invention includes, as a thirteenth aspect, a system according to the eleventh or twelfth aspect, wherein: the display unit displays a fourth interface screen that includes a graph including a distribution chart indicating a distribution of samples in a population and the reference range on a same coordinate plane, the population being generated based on the datasets for assessment, the fourth interface screen receiving an operation carried out by a user on the graph; and the display unit displays information on a sample specified by the user via the fourth interface screen.

The present invention includes, as a fourteenth aspect, a program causing a computer to execute: a first acquiring step for acquiring a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee; a generating step for extracting from the first database datasets that satisfy the first extraction condition, and generating a second database that stores the datasets extracted from the first database; a storing step for storing the second database; a second acquiring step for acquiring a second extraction condition dataset indicating a second extraction condition for extracting datasets from the second database, the second extraction condition relating to at least one of an item of extraction, a period of examination, and an attribute of an examinee; a third acquiring step for acquiring a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions; an extraction step for extracting from the second database datasets that satisfy the second extraction condition; a specifying step for specifying a reference range within which samples that are not rejected from a population in accordance with the rejection rate are distributed, the population being generated based on the datasets extracted from the second database and consisting of samples, each of which indicates a result of the first examination and a result of the second examination; and a displaying step for displaying a graph with a first coordinate axis representing a result of the first examination and a second coordinate axis representing a result of the second examination, the graph indicating the reference range.

The present invention includes, as a fifteenth aspect, a method executed by a data processing device comprising: a first acquiring step for acquiring a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee; a generating step for extracting from the first database datasets that satisfy the first extraction condition, and generating a second database that stores the datasets extracted from the first database; a storing step for storing the second database; a second acquiring step for acquiring a second extraction condition dataset indicating a second extraction condition for extracting datasets from the second database, the second extraction condition relating to at least one of an item of extraction, a period of examination, and an attribute of an examinee; a third acquiring step for acquiring a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions; an extraction step for extracting from the second database datasets that satisfy the second extraction condition; a specifying step for specifying a reference range within which samples that are not rejected from a population in accordance with the rejection rate are distributed, the population being generated based on the datasets extracted from the second database and consisting of samples, each of which indicates a result of the first examination and a result of the second examination; and a displaying step for displaying a graph with a first coordinate axis representing a result of the first examination and a second coordinate axis representing a result of the second examination, the graph indicating the reference range.

### Effects of the Invention

According to the present invention, a burden imposed on an operator in determining a reference range for assessing appropriateness of clinical examination results is reduced.

### BRIEF EXPLANATION OF THE DRAWINGS

FIG. 1 illustrates a configuration of a medical data processing system according to an exemplary embodiment of the present invention.

FIG. 2 illustrates a hardware configuration of a medical data processing device according to the exemplary embodiment of the present invention.

FIG. 3 illustrates a functional configuration of the medical data processing device according to the exemplary embodiment of the present invention.

FIG. 4 illustrates a whole processing flow carried out by the medical data processing system.

FIG. 5 illustrates a processing flow from a step for extracting datasets of examination results to a step for storing the extracted datasets.

FIG. 6 illustrates a processing flow for generating a distribution chart with a reference range.

FIG. 7 illustrates a processing flow for assessing whether an examination result indicated by each of the extracted datasets is within the reference range.

FIG. 8 illustrates a processing flow for specifying the reference range.

FIG. 9 illustrates a processing flow for assessing whether an examination result acquired from an analyzer is within the reference range.

FIG. 10 illustrates an example of a distribution chart.

FIG. 11 illustrates an example of a reference range in connection with a relationship between results of a previous examination and a current examination.

FIG. 12A illustrates how the reference range is specified in connection with the relationship between results of the previous examination and the current examination.

FIG. 12B illustrates how the reference range is specified in connection with the relationship between the results of the previous examination and the current examination.

FIG. 12C illustrates how the reference range is specified in connection with a relationship between results of two different types of examination.

FIG. 12D illustrates how the reference range is specified in connection with a relationship between the results of two different types of examination.

FIG. 13A illustrates an example of a screen for displaying distribution charts and a result of assessment of the distribution charts.

FIG. 13B illustrates Areas 11 and 13 of the screen.

FIG. 13C illustrates Areas 15 and 16 of the screen.

FIG. 13D illustrates Area 13 of the screen.

FIG. 13E illustrates a screen for displaying information about a selected examinee.

FIG. 13F illustrates Areas 14 and 13 of the screen.

FIG. 13G illustrates Areas 12 and 13 of the screen.

FIG. 13H illustrates Areas 13 and 17 of the screen.

FIG. 14 illustrates a screen for displaying results of validity assessments of the examination results.

### MODES FOR CARRYING OUT THE INVENTION

### [1] Examples

### (System Configuration)

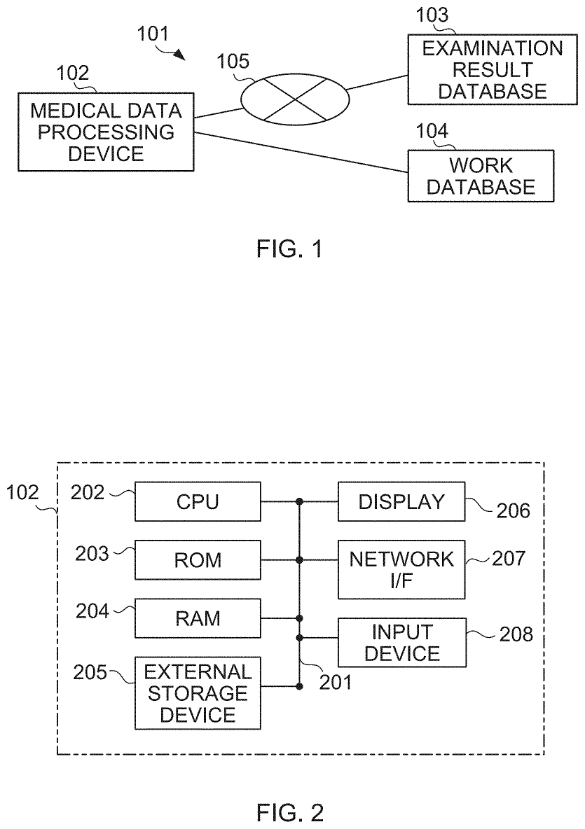

FIG. 1 illustrates a configuration of a medical data processing system according to an exemplary embodiment of the present invention. As illustrated in FIG. 1, Medical Data Processing System 101 comprises Medical Data Processing Device 102, Examination Result Database 103, and Work Database 104. Examination Result Database 103 stores examination result datasets, each of which indicates a result of clinical examination relating to an examinee, i.e. a patient.

More specifically, Examination Result Database 103 stores datasets that each relate to a clinical examination, and indicate an item of examination, a value of examination result, a date of examination, an identifier of an examinee, and one or more attributes of the examinee. Examination Result Database 103 is an example of a first database described in the claims of the present application.

The one or more attributes of the examinee may include, for example, inpatient/outpatient classification, gender, age, hospital department, hospital ward, presence/absence of dialysis, and the like. Examination Result Database 103 may be provided either in Medical Data Processing Device 102 or in a data processing device other than Medical Data Processing Device 102. In general, Medical Data Processing Device 102 is provided in a database server of the system for clinical examination.

Work Database 104 stores datasets extracted from Examination Result Database 103 in accordance with predetermined extraction conditions. Each of the datasets stored in Work Database 104 indicates an item of examination, a date of examination, an identifier of examination, an identifier of an examinee, one or more attributes of the examinee, a value of examination result, etc. Work Database 104 is an example of a second database described in the claims of the present application.

Medical Data Processing Device 102 extracts datasets from Examination Result Database 103 via Network 105, and stores the extracted datasets in Work Database 104. Medical Data Processing Device 102 also extracts datasets from Examination Result Database 103 or Work Database 104, and performs data processing using the extracted datasets in response to a request from a data processing device other than Medical Data Processing Device 102 included in Medical Data Processing System 101. Further, Medical Data Processing Device 102 transmits datasets indicating results of the data processing to a data processing device connected to Medical Data Processing System 101.

### (Hardware Configuration of Medical Data Processing Device 102)

FIG. 2 illustrates a hardware configuration of Medical Data Processing Device 102 according to the present exemplary embodiment. Medical Data Processing Device 102 comprises CPU (Central Processing Unit) 202, ROM (Read-Only Memory) 203, RAM (Random Access Memory) 204, External Storage Device 205 that may include a HDD (hard disk drive) or a SSD (solid state drive), Display 206, Network I/F (interface) 207, and Input Device 208 that may include a keyboard, a mouse and the like.

Each of the components from CPU 202 to Input Device 208 is connected to Internal Bus 201. CPU 202 controls all of the components of Medical Data Processing Device 102. ROM 203 stores a boot program for starting Medical Data Processing Device 102. RANI 204 is used as a work area of CPU 202. External Storage Device 205 controls reading and writing of data, and stores programs for processing medical data and various types of data such as Work Database 104 illustrated in FIG. 1.

External Storage Device 205 is an example of a storage unit described in the claims of the present application. As well as Work Database 104, External Storage Device 205 may store Examination Result Database 103. Display 206 is, for example, a display unit of Medical Data Processing Device 102 illustrated in FIG. 1, and displays various types of information. Network I/F 207 controls data communications between Medical Data Processing Device 102 and a data processing device other than Medical Data Processing Device 102. Input Device 208 is a device that enables an operator to input data to Medical Data Processing Device 102.

### (Functional Configuration of Medical Data Processing Device 102)

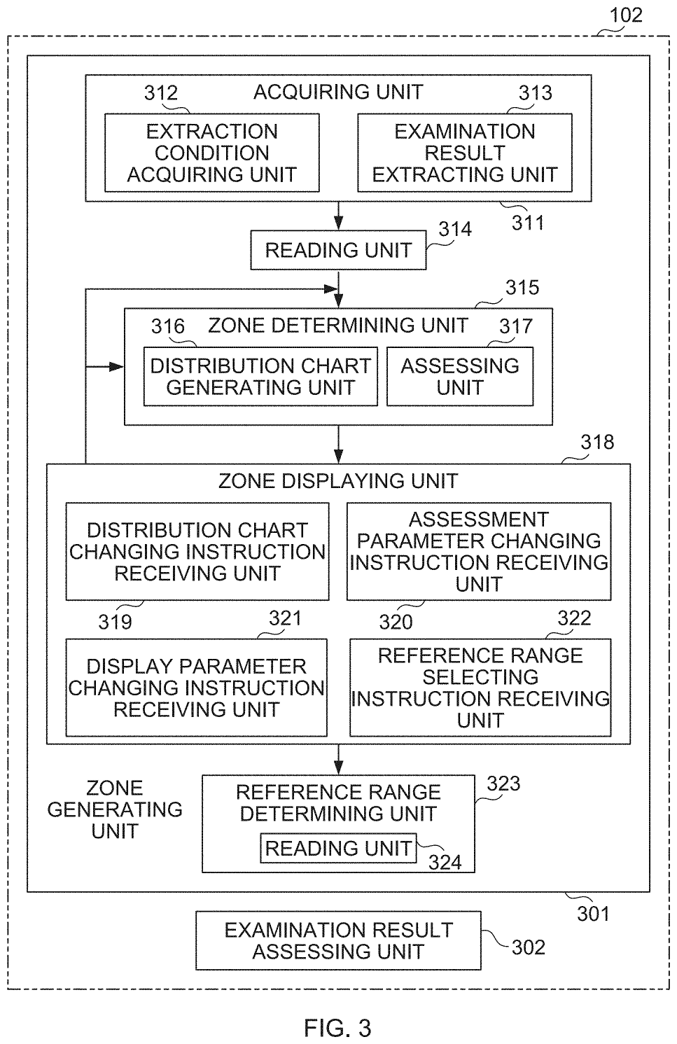

FIG. 3 illustrates a functional configuration of Medical Data Processing Device 102 according to the present exemplary embodiment. Medical Data Processing Device 102 comprises Zone Generating Unit 301 and Examination Result Assessing Unit 302. Zone Generating Unit 301 includes Acquiring Unit 311, Reading Unit 314, Zone Determining Unit 315, Zone Displaying Unit 318, and Reference Range Determining Unit 323.

Acquiring Unit 311 includes Extraction Condition Acquiring Unit 312 and Examination Result Extracting Unit 313. Extraction Condition Acquiring Unit 312 acquires a dataset indicating conditions for extracting datasets from Examination Result Database 103 in response to operations made by an operator via a screen of Medical Data Processing Device 102. The conditions for extracting datasets from Examination Result Database 103 are hereinafter referred to as first extraction conditions, and the dataset indicating the first extraction conditions are hereinafter referred to as first extraction condition dataset.

The first extraction conditions are examples of a first extraction condition described in the claims of the present application, and the first extraction condition dataset is an example of first extraction condition dataset described in the claims of the present application. Extraction Condition Acquiring Unit 312 is an example of a first acquiring unit described in the claims of the present application. Extraction Condition Acquiring Unit 312 acquires a first extraction condition dataset that indicates first extraction conditions relating to at least one of an item of examination, a period of examination, and one or more attributes of an examinee.

In the present exemplary embodiment, Extraction Condition Acquiring Unit 312 acquires, as a first extraction condition dataset, a dataset indicating each of an item of examination, a period of examination, and plural attributes of an examinee such as inpatient/outpatient classification, gender, age, hospital department, hospital ward, presence/absence of dialysis, etc. Examination Result Extracting Unit 313 extracts from Examination Result Database 103 datasets that satisfy the first extraction conditions indicated by the first extraction condition dataset acquired by Extraction Condition Acquiring Unit 312.

Examination Result Extracting Unit 313 generates Work Database 104 for storing the extracted datasets, and instructs External Storage Device 205 to store Work Database 104. Examination Result Extracting Unit 313 is an example of a generating unit described in the claims of the present application. External Storage Device 205 stores Work Database 104 generated by Examination Result Extracting Unit 313.

Reading Unit 314 repeatedly reads out datasets from Work Database 104 in response to requests made by Zone Determining Unit 315. Zone Determining Unit 315 determines a zone displayed on a distribution chart that indicates a correlation of two groups of examination results indicated by the datasets read by Reading Unit 314.

A zone is an area of a distribution chart for determining whether an examination-result value is likely normal or abnormal. In other words, a zone is an area for indicating a reference range of a value that has a high possibility of being normal, or of a value that is acceptable as a normal value. Thus, an examination-result value shown within a zone on a distribution chart is accepted as a normal value, and an examination-result value shown outside the zone on the distribution chart is rejected as an abnormal value. Specific examples of distribution charts and zones in the charts will be described later.

Zone Determining Unit 315 includes Distribution Chart Generating Unit 316 and Assessing Unit 317. Distribution Chart Generating Unit 316 generates a distribution chart with a zone by using datasets read from Work Database 104 by Reading Unit 314. Assessing Unit 317 assesses each of values of examination results shown in the distribution chart generated by Distribution Chart Generating Unit 316 using datasets extracted by Examination Result Extracting Unit 313.

Zone Displaying Unit 318 displays a distribution chart with a zone determined by Zone Determining Unit 315 and results of assessment performed by Assessing Unit 317 on a screen of Display 206 of Medical Data Processing Device 102. Zone Displaying Unit 318 is an example of a display unit described in the claims of the present application. Zone Displaying Unit 318 receives instructions provided by the operator for modifying a zone in a distribution chart, modifying parameters for displaying a zone, modifying parameters for evaluating examination results, and generating a reference range, etc.

Zone Displaying Unit 318 includes Distribution Chart Changing Instruction Receiving Unit 319, Assessment Parameter Changing Instruction Receiving Unit 320, Display Parameter Changing Instruction Receiving Unit 321, and Reference Range Selecting Instruction Receiving Unit 322. Distribution Chart Changing Instruction Receiving Unit 319 receives instructions provided by the operator for changing the distribution chart to be displayed. When a plurality of sets of the first extraction conditions are selected by the operator, Distribution Chart Generating Unit 316 generates a plurality of distribution charts corresponding to the plurality of sets of the first extraction conditions. When the operator provides operations to Medical Data Processing Device 102 for selecting a distribution chart to be newly displayed from among the plurality of distribution charts, Distribution Chart Changing Instruction Receiving Unit 319 receives an instruction for changing the distribution chart to be displayed.

Assessment Parameter Changing Instruction Receiving Unit 320 receives instructions for changing a parameter for assessment. The parameter for assessment is a ratio of a portion of the aforementioned distribution chart to the whole distribution chart that is rejected, since examination-result values in the portion are highly likely to be abnormal. The ratio is also referred to as a “rejection rate.” Display Parameter Changing Instruction Receiving Unit 321 receives instructions for changing parameters for displaying a distribution chart. The parameters for displaying the distribution chart may include a width of a display area of the distribution chart, a representation form of axes of the distribution chart, etc.

Reference Range Selecting Instruction Receiving Unit 322 selects a reference range for the above-described assessment, i.e., assessment of whether the examination result indicates a normal value or an abnormal value. The reference range is a range excluding a portion rejected by the rejection rate described above, and also constitutes a condition for generating a zone. For example, if the rejection rate is 5%, the reference range is 95%. In this case, the condition for generating a zone may be either 95% (reference range) or 5% (rejection rate).

Reference Range Determining Unit 323 includes Reading Unit 324. Reading Unit 324 reads examination result datasets stored in Work Database 104 that meet the conditions received by Reference Range Selecting Instruction Receiving Unit 322. Reference Range Determining Unit 323 generates a reference range dataset that indicates a reference range, i.e. a range of examination results indicated by the examination result datasets read by Reading Unit 324. Examination Result Assessing Unit 302 acquires an examination result from the analyzer, and assesses the examination result based on the reference range dataset generated by Reference Range Determining Unit 323.

### (Procedure for Processing Medical Data)

Next, a procedure for processing medical data executed by Medical Data Processing Device 102 will be described.

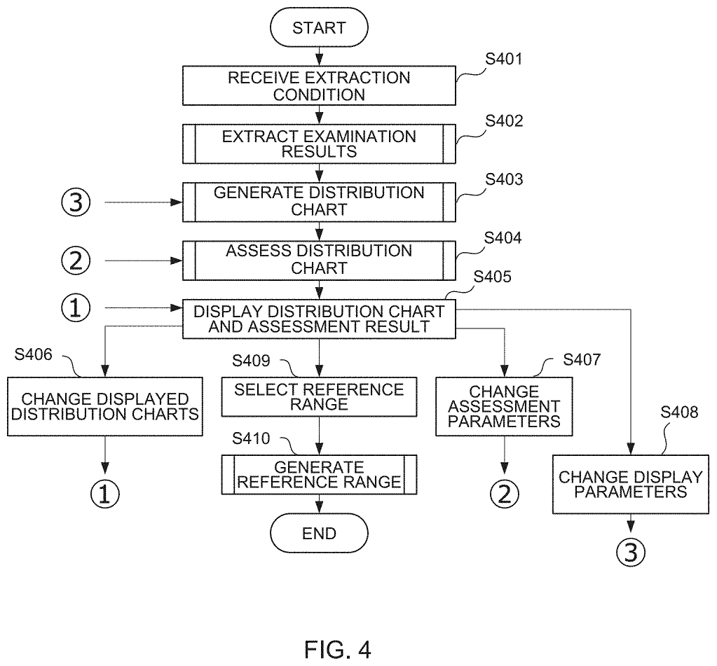

FIGS. 4 to 9 illustrate processing flows executed by Medical Data Processing Device 102. FIG. 4 (steps S401 to S410) illustrates the entire processing flow for generating a zone. This processing flow includes each of steps from a step for extracting examination result datasets from Examination Result Database 103 based on predetermined conditions to a step for generating a reference range dataset.

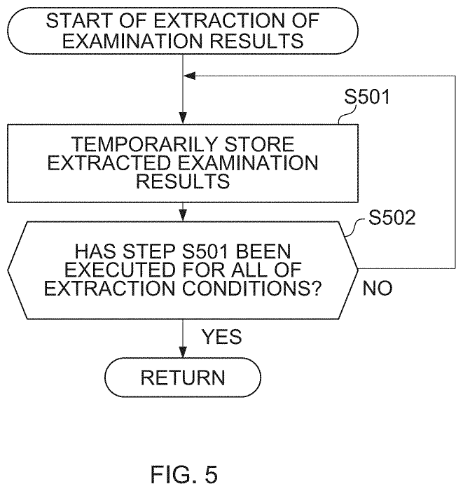

FIG. 5 illustrates a processing flow including each of steps from a step for extracting examination result datasets to a step for storing the extracted datasets. The processing flow of FIG. 5 illustrates detailed steps constituting step S402 in FIG. 4. The processing flow of FIG. 5 includes each of steps from a step for extracting examination result datasets to a step for generating Work Database 104 as a temporary file that stores the extracted examination result datasets for each extraction condition.

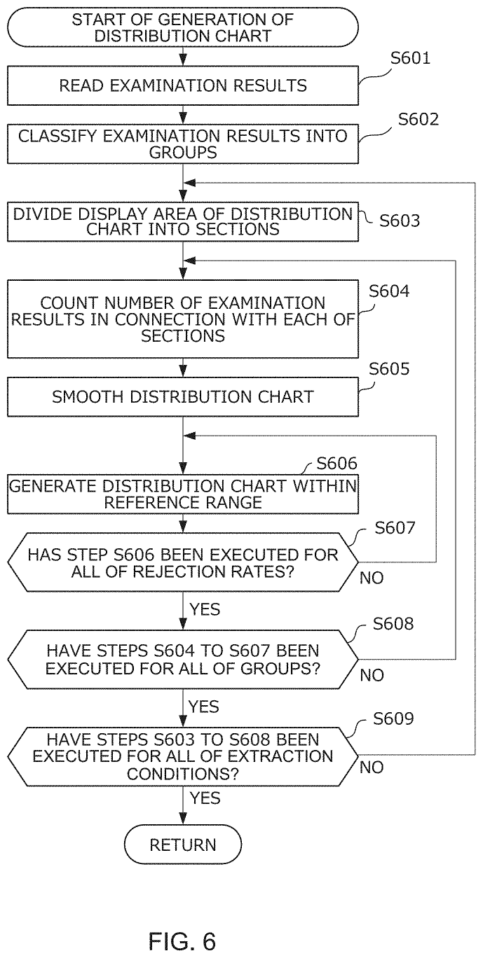

FIG. 6 illustrates a processing flow for determining a reference range. The processing flow illustrated in FIG. 6 includes detailed steps constituting step S403 in FIG. 4. The processing flow illustrated in FIG. 6 includes steps for generating a reference range dataset indicating a reference range used for assessing whether each examination result indicates a normal value or an abnormal value.

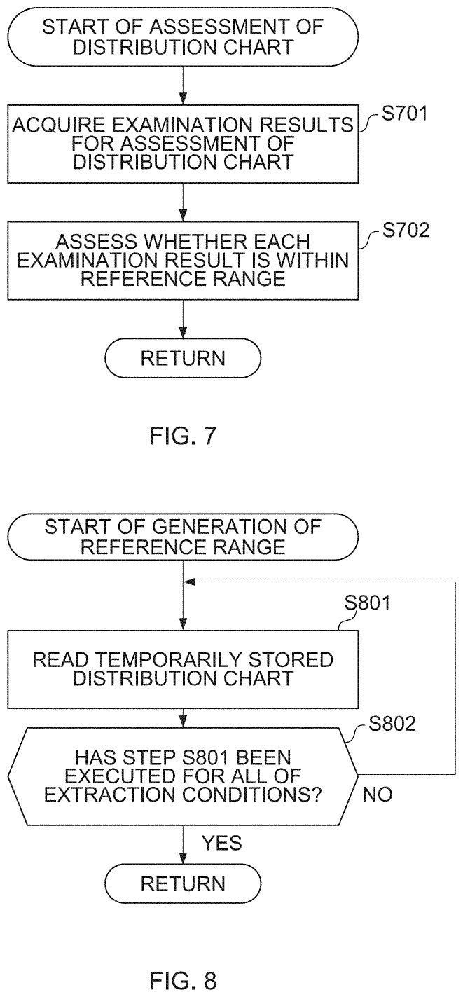

FIG. 7 illustrates a processing flow for assessing whether each of the extracted examination results falls within the reference range. The processing flow illustrated in FIG. 7 includes detailed steps constituting step S404 in FIG. 4. The processing flow illustrated in FIG. 7 includes steps for assessing whether each of the examination results extracted by Examination Result Extracting Unit 313 of Acquiring Unit 311 falls within a reference range when the reference range is determined by use of a rejection rate in connection with a selected distribution chart.

FIG. 8 illustrates a processing flow for generating a reference range dataset. The processing flow illustrated in FIG. 8 includes detailed steps constituting step S410 in FIG. 4. The processing flow illustrated in FIG. 8 includes steps for reading a distribution chart dataset and for generating a reference range dataset. As described above, FIGS. 5 to 8 illustrate details of the processing flow in FIG. 4.

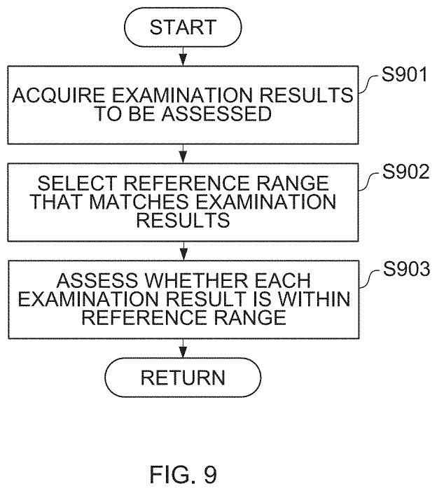

FIG. 9 illustrates a processing flow for using a zone determined in the processing following the flow of FIG. 4. More specifically, FIG. 9 illustrates a processing flow for assessing whether an examination result acquired from the analyzer is within the reference range. FIG. 9 illustrates a processing flow that includes steps for acquiring an examination result from the analyzer, for selecting a reference range from among candidate reference ranges indicated by reference range datasets generated by Reference Range Determining Unit 323, and for assessing whether the examination result is within the selected reference range.

Each of the steps in FIG. 4 will now be described. Step S401 is a step for receiving an extraction condition. At step S401, Extraction Condition Acquiring Unit 312 of Medical Data Processing Device 102 acquires a first extraction condition dataset that indicates a first extraction condition for extracting datasets relating to examination. Extraction Condition Acquiring Unit 312 of Medical Data Processing Device 102 acquires a first extraction condition dataset indicating a first extraction condition relating to, for example, an item of examination, a period of examination, and one or more attributes of an examinee. The first extraction condition dataset may indirectly indicate the first extraction condition by indicating examination results that should be eliminated upon extraction. In this case, the examination results that should be eliminated may be indicated by reception numbers of examination, identification numbers of examinees, etc.

Step S402 is a step for extracting examination results and for storing the extracted examination results. At step S402, Examination Result Extracting Unit 313 of Medical Data Processing Device 102 extracts from Work Database 104 datasets that meet the first extraction condition, and stores the extracted datasets in Work Database 104. Step S402 will be described in detail later with reference to FIG. 5. Step S403 is a step for generating a distribution chart dataset. Step S403 will be described in detail later with reference to FIG. 6.

Step S404 is a step for performing an assessment. Step S404 is carried out after step S403 for generating a distribution chart dataset, or after step S407 for receiving an instruction to change an assessment parameter. Step S404 will be described in detail later with reference to FIG. 7.

Step S405 is a step for reading a distribution chart dataset and an assessment result dataset, and for displaying a distribution chart and an assessment result indicated by the datasets. At step S405, Zone Displaying Unit 318 of Medical Data Processing Device 102 reads the distribution chart dataset generated at step S403 and an assessment result dataset indicating a result of the assessment carried out at step S404, and displays a distribution chart indicated by the distribution chart dataset and an assessment result indicated by the assessment result dataset on Display 206 of Medical Data Processing Device 102. Step S405 is carried out after step S404 for performing the assessment, or after step S406 for receiving an instruction to change distribution charts to be displayed. At Step 405, a distribution chart and a result of assessment corresponding to each extraction condition are displayed.

Step S406 is a step for receiving an instruction to change distribution charts to be displayed. At step S406, Zone Displaying Unit 318 of Medical Data Processing Device 102 receives an instruction to change zones to be displayed on Display 206 for consideration for selection by an operator. The received instruction is used at step S405 to refresh the displayed information.

Step S407 is a step for receiving an instruction to change parameters for assessment. When an operator changes rejection rates via a screen displayed by Display 206 to change parameters and instruct refresh of a currently displayed zone, Zone Displaying Unit 318 of Medical Data Processing Device 102 receives the instruction provided by the operator as an instruction to change parameters for assessment at step S407. The instruction to change parameters received at step S407 is used at step S404 to perform a new assessment. Then, a new zone for consideration for selection by the operator is displayed at step S405.

Step S408 is a step for receiving an instruction to change parameters for displaying a distribution chart. At step S408, Zone Displaying Unit 318 of Medical Data Processing Device 102 receives an instruction to change parameters for displaying a zone for consideration displayed by Display 206 such as a width of area for displaying a distribution chart, a type of axes of the distribution chart, a boundary number of elapsed days for the distribution chart, etc. According to the instruction received at step S408, a new distribution chart is generated at step S403. Namely, the change of parameters for displaying a zone corresponds to a change of conditions for generating a zone.

Step S409 is a step for receiving a selection of a reference range. At step S409, Zone Displaying Unit 318 of Medical Data Processing Device 102 receives a reference range dataset that indicates a selected reference range used for assessing whether each of examination results shown in a zone currently displayed by Display 206 indicates an abnormal value.

Step S410 is a step for generating a reference range dataset. At step S410, Reference Range Determining Unit 323 of Medical Data Processing Device 102 reads from Work Database 104 a distribution chart dataset, and generates a reference range dataset indicating a new reference range to be displayed in the currently displayed zone that is generated following receipt of new parameters for displaying the zone, i.e. the new conditions for generating the zone. Step S410 will be described in detail later with reference to FIG. 9. After execution of step S410, the operator determines an optimal zone for use in assessing examination results. The processing flow in FIG. 4 is completed at step S410.

Each of the steps in FIG. 5 will now be described. At step S501, Examination Result Extracting Unit 313 of Medical Data Processing Device 102 temporarily stores in Work Database 104, for each of first extraction conditions, extracted datasets that meet the first extraction conditions. At step S502, Examination Result Extracting Unit 313 judges whether extraction has been executed in accordance with each of first extraction conditions, which may relate to an item of examination, a period of examination (for generating a reference range and for evaluating examination results), an attribute of examinee, etc. While these steps are repeatedly executed, extracted examination result datasets for each of plural first extraction conditions are stored in Work Database 104.

Each step in FIG. 6 will be now be described. At step S601, Reading Unit 314 of Medical Data Processing Device 102 reads datasets that meet a specified extraction condition from among datasets stored in Work Database 104. The specified extraction condition used at step S601 is hereinafter referred to as a “second extraction condition,” and a dataset indicating a second extraction condition is hereinafter referred to as a “second extraction condition dataset.”

The “second extraction condition” is an example of the “second extraction condition” described in the claims of the present application, and the “second extraction condition dataset” is an example of the “second extraction condition dataset” described in the claims of the present application. In the present embodiment, the second extraction condition dataset is acquired by Display Parameter Changing Instruction Receiving Unit 321. Display Parameter Changing Instruction Receiving Unit 321 receives an instruction to change parameters for displaying a distribution chart.

The parameters for displaying a distribution chart function as the second extraction condition that is used for extracting examination result datasets for generating a distribution chart for display. Display Parameter Changing Instruction Receiving Unit 321 is an example of “second acquiring unit” described in the claims of the present application. The parameters for displaying a distribution chart may include, as a second extraction condition, a parameter relating to an item of examination such as “sodium concentration in serum,” a parameter relating to a period of examination such as “last year,” a parameter relating to an attribute of an examinee such as “outpatient.” The parameters may include parameters relating to two or more different types of attributes of the examinee.

Reading Unit 314 extracts from Work Database 104 datasets meeting the second extraction condition indicated by the second extraction condition dataset acquired by Display Parameter Changing Instruction Receiving Unit 321. Reading Unit 314 is an example of the “extraction unit” described in the claims of the present application.

At step S602, Distribution Chart Generating Unit 316 of Medical Data Processing Device 102 classifies the datasets read by Reading Unit 314 at step S601 into one of groups divided by a specified number of days based on the number of days that has elapsed from a previous examination to the examination whose result is indicated by the dataset. At step S603, Distribution Chart Generating Unit 316 of Medical Data Processing Device 102 divides the display area of a distribution chart into plural sections in accordance with the predetermined conditions such that information displayed in the display area can be viewed with ease by the operator.

Step S603 is also executed after step S609 is executed. At step S604, Distribution Chart Generating Unit 316 of Medical Data Processing Device 102 counts, in connection with each of the sections of the distribution chart, a number of datasets that meet the condition corresponding to the section, and temporarily stores a dataset indicating a result of the count for each section together with datasets indicating summarized information of the extracted examination results indicated by the extracted datasets. The summarized information of the extracted examination results may include values of the examination results, reception dates of the examinations, reception numbers of the examinations, etc. Step S604 is repeatedly executed for each of the groups divided at step S601 until a result of the judgment at step S608 described later becomes positive.

At step S605, Distribution Chart Generating Unit 316 of Medical Data Processing Device 102 executes smoothing processing on the distribution chart. The smoothing processing is executed to make the characteristics of the zone easier for the operator to understand. At step S606, Distribution Chart Generating Unit 316 of Medical Data Processing Device 102 specifies sections of the distribution chart that are within the reference range, and generates a distribution chart consisting only of the sections that are within the reference range. Step S606 is repeatedly executed for each of rejection rates until a result of the assessment at step S607 described later becomes positive.

Distribution Chart Generating Unit 316 specifies the reference range based on a rejection rate that is received by Assessment Parameter Changing Instruction Receiving Unit 320 as a parameter for assessment. Assessment Parameter Changing Instruction Receiving Unit 320 receives an instruction to change the parameter for assessment made by the operator, and acquires a rejection rate indicated by the instruction. Distribution Chart Generating Unit 316 is an example of a “specifying unit” described in the claims of the present application, and Assessment Parameter Changing Instruction Receiving Unit 320 is an example of a “third acquiring unit” described in the claims of the present application.

Steps S607 to S609 are steps for splitting the processing flow. Step S607 is a step for causing step S606 to be executed for all of the rejection rates. Step S608 is a step for causing steps S604 to S607 to be executed for all of the groups divided by the number of elapsed days. Step S609 is a step for causing steps S603 to S608 to be executed for all of the extraction conditions. Distribution charts each indicating a reference range are generated by the above described steps in FIG. 6.

As described above, at step S403 in FIG. 4, Distribution Chart Generating Unit 316 of Medical Data Processing Device 102 generates distribution charts by use of datasets extracted from Work Database 104 by Reading Unit 314. The operator is able to view the distribution chart generated in accordance with a new parameter for displaying the distribution chart simply by instructing a change of the parameters, and can thus efficiently obtain information for use in assessing whether each examination-result value is normal.

Each step in FIG. 7 will now be described. At step S701, Assessing Unit 317 of Medical Data Processing Device 102 acquires datasets for assessment of distribution chart that are extracted by use of extraction conditions provided by the operator. At step S702, Assessing Unit 317 of Medical Data Processing Device 102 refers to the distribution chart indicating the reference range, and assesses, for each of the datasets acquired at step S701, whether an examination result indicated by the dataset is within the reference range.

Each step in FIG. 8 will now be described. At step S801, Reading Unit 324 of Medical Data Processing Device 102 reads the dataset indicating the distribution chart that is temporarily stored in Work Database 104. By reading the dataset indicating the temporarily stored distribution chart, Medical Data Processing Device 102 can perform the processing faster than by generating the dataset again. At step S802, Reference Range Determining Unit 323 of Medical Data Processing Device 102 repeatedly executes processing to generate a reference range for each of the extraction conditions until a dataset is generated for all of the extraction conditions. Step S802 is a step for causing step S801 to be executed for all of the extraction conditions. Following the flow of FIG. 8, Medical Data Processing Device 102 can quickly acquire datasets indicating a reference range for all of the extraction conditions.

In the above, a processing flow for generating a zone executed by Zone Generating Unit 301 is described. Following is a description of a processing flow illustrated in FIG. 9 for assessing examination results by use of the zone generated by Zone Generating Unit 301. At step S901, Examination Result Assessing Unit 302 of Medical Data Processing Device 102 acquires a dataset indicating an examination result to be assessed. The dataset acquired by Examination Result Assessing Unit 302 at step S901 is a dataset acquired from the analyzer.

At step S902, Examination Result Assessing Unit 302 of Medical Data Processing Device 102 compares attributes of the examination result with attributes of reference ranges and selects a reference range whose attributes match those of the examination result. Step S902 is a step for searching a reference range from among the stored reference ranges, i.e. zones, that is appropriate as an examination result from a viewpoint of examination-result attributes, such as whether the examinee is an outpatient or an inpatient, gender of the examinee, age of the examinee, etc. At step S903, Examination Result Assessing Unit 302 of Medical Data Processing Device 102 assesses whether the examination result acquired at step S901 is within the reference range selected at step S902. At step S903, Examination Result Assessing Unit 302 recognizes the examination result as a valid examination result when the examination result is within the reference range, and recognizes the examination result as an invalid examination result when the examination result is outside the reference range.

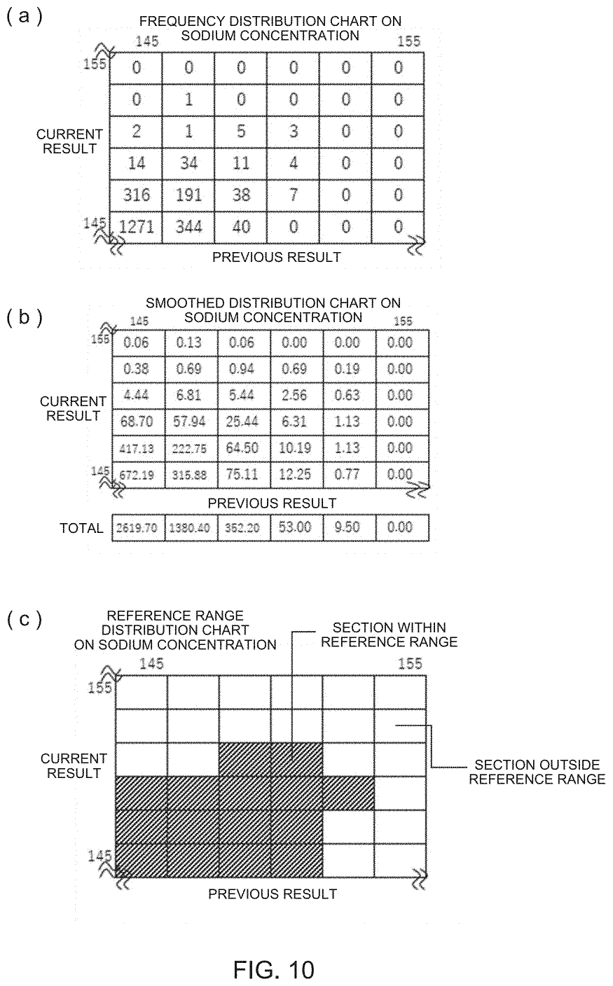

FIG. 10 illustrates an example of a distribution chart. FIG. 10(a) illustrates a part of an example of a frequency distribution chart. The frequency distribution chart is generated using the datasets indicating examination results read from Work Database 104, and indicates a distribution of frequency of examination results for sodium concentration in serum. The vertical axis of the chart represents a value of a current examination result, and the horizontal axis of the chart represents a value of a previous examination result.

Here, “current examination result” is an examination result to be assessed, and may be a result of an examination performed recently for an examinee, or a result of examination performed within a previous time period for the examinee. Here, “previous examination result” means a result of the same type of examination performed at a concurrently previous time for the same examinee. The time interval between the current examination and the previous examination need not be constant.

Distribution Chart Generating Unit 316 generates a distribution chart having a display area that is composed of sections arranged in two dimensions obtained by dividing the display area into an equal number of sections in the vertical axis direction and in the horizontal axis direction, as illustrated, for example, in FIG. 10(a). The distribution chart illustrated in FIG. 10(a) has a display area having, for example, 40 sections in the vertical axis direction and 40 sections in the horizontal axis direction, although only 6 sections in each of the vertical and horizontal directions are shown in FIG. 10(a). The number of sections in each of the vertical and horizontal directions is not limited to 40, and an appropriate number is adopted for each case. This is because a number of examination results corresponding to each section may be too small if the number of sections in each direction is too large, and accuracy of assessing a validity of an examination result using the distribution chart will deteriorate if the number of sections in each direction is too small.

At step S601, Reading Unit 314 reads datasets indicating examination results for sodium concentration in serum from Work Database 104. At step S602, Distribution Chart Generating Unit 316 classifies the read datasets into plural groups based on, for example, a number of days that has elapsed from a previous examination, when Extraction Condition Acquiring Unit 312 acquires a first extraction condition relating to number of days that has elapsed from the previous examination.

At step S603, Distribution Chart Generating Unit 316 specifies, for each of the groups of extracted datasets, a maximum value and a minimum value for the examination results in the group, generates a display area of a distribution chart whose ranges in the vertical axis direction and the horizontal axis direction are between the maximum and minimum values, and divides the display area into plural sections. As explained above, FIG. 10(a) illustrates a part of an example of a distribution chart having 40×40 sections, i.e. 40 sections in the vertical axis direction and 40 sections in the horizontal axis direction. At step S604, Distribution Chart Generating Unit 316 counts a number of examination results corresponding to each of the sections, and temporarily stores a dataset indicating the count result for each section together with a dataset indicating summary information for each of the examination results.

FIG. 10(b) illustrates a part of an example of a smoothed distribution chart. Distribution Chart Generating Unit 316 generates the smoothed distribution chart by applying the smoothing process to the frequency distribution chart illustrated in FIG. 10(a). In the frequency distribution chart generated from actual examination results, the values of adjacent sections may vary unnaturally. In this embodiment, in order for the distribution chart to show a more natural change in values of adjacent sections, the smoothing process is applied to the frequency distribution chart such as shown in the chart illustrated in FIG. 10(a).

Partial deviation or the like may occur in the number of examination results distributed into the sections. Therefore, it is assumed that values in the distribution chart follow a Gaussian distribution, and Distribution Chart Generating Unit 316 calculates a new value for each section by distributing a portion of a current value of each section to its adjacent sections. For example, the following matrix is used for the smoothing processing.

$$( \begin{matrix}1 \text{/} 16 & 2 \text{/} 16 & 1 \text{/} 16 \\ 2 \text{/} 16 & 4 \text{/} 16 & 2 \text{/} 16 \\ 1 \text{/} 16 & 2 \text{/} 16 & 1 \text{/} 16\end{matrix} )$$

Distribution Chart Generating Unit 316 distributes, for each section of the original distribution chart, a value of the target section to the adjacent sections of the target section and the target section itself following the proportions shown in the matrix. Assuming that R(n,m) is a value of a section of the n-th in the x-axis direction and the m-th in the y-axis direction of the original distribution chart, and S(n,m) is a value of a section of the n-th in the x-axis direction and the m-th in the y-axis direction of the smoothed distribution chart, S(m,n) is expressed as follows:

S(n,m)= 1/16(R(n−1,m−1))+ 2/16(R(n,m−1))+ 1/16(R(n+1,m−1))+ 2/16(R(n−1,m))+ 4/16(R(n,m))+ 2/16(R(n+1,m))+ 1/16(R(n−1,m+1))+ 2/16(R(n,m+1)+ 1/16(R(n+1,m+1)).

At step S605, Distribution Chart Generating Unit 316 calculates S(n,m) and generates a smoothed distribution chart using the calculated S(n,m) in the same format as in the original distribution chart. FIG. 10(c) illustrates a part of an example of a reference range distribution chart. Distribution Chart Generating Unit 316 generates the reference range distribution chart by applying rejection processing to the smoothed distribution chart illustrated in FIG. 10(b). Distribution Chart Generating Unit 316 determines, for each group of sections of the n-th in the x-axis showing a previous value, an upper limit section, and a lower limit section in a y-axis showing a current value, so that a ratio of the total value of sections outside the range between the upper limit section and the lower limit section to the total value of all sections in the smoothed distribution chart becomes the given rejection rate.

At step S606, Distribution Chart Generating Unit 316 generates a zone based on comparison with a previous value, which is hereinafter referred to as a “previous value comparison zone,” by excluding sections that do not fall within the range between the lower limit section and the upper limit section, and by further excluding sections whose value is less than 1. A previous value comparison zone is a distribution chart for assessing an examination result from a viewpoint of comparison with a previous result of the same type of examination performed for the same examinee. Namely, a rejection rate used for generating a previous value comparison zone is a ratio of a number of samples to be rejected to a number of total samples in a population from a viewpoint of comparison with a previous result of the same type of examination performed for the same examinee.

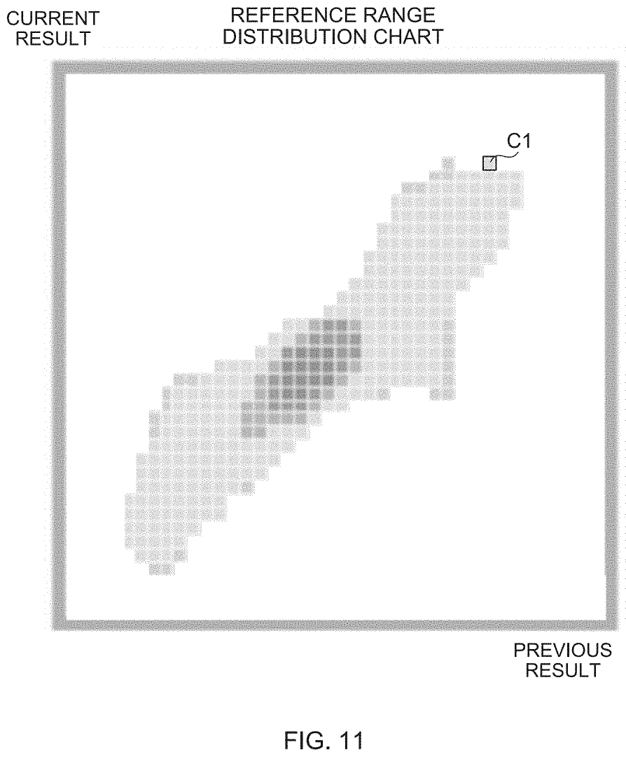

FIG. 11 illustrates an example of a previous value comparison zone. In the zone illustrated in FIG. 11, each section is represented by a square, and one section is marked with sign C1. In the zone illustrated in FIG. 11, a shade of a section becomes darker with an increase in its value. In the previous value comparison zone, a relationship between a current examination result and a previous examination result is depicted by a difference in shading of sections. Distribution Chart Generating Unit 316 may generate a different type of zone. For example, Distribution Chart Generating Unit 316 generates a zone for assessing an examination result from a viewpoint of correlation with a result of a different type of examination performed for the same examinee at the same time. This zone is hereinafter referred to as an “inter-item correlation zone.” Distribution Chart Generating Unit 316 generates an inter-item correlation zone in a manner similar to that for generating a previous value comparison zone. Namely, Distribution Chart Generating Unit 316 excludes sections from a smoothed distribution chart with an x-axis representing a result of a first type of examination and a y-axis representing a result of a second type of examination so that a ratio of the total value of remaining sections to the total value of all sections becomes a given rejection rate.

When Distribution Chart Generating Unit 316 generates an inter-item correlation zone at step S606, Distribution Chart Generating Unit 316 determines a threshold larger than 1 so that a ratio of the total value of sections whose value is less than the threshold to the total value of all sections becomes a given rejection rate. As described above, Distribution Chart Generating Unit 316 generates a zone indicating a reference range for assessing whether an examination result is normal by excluding some samples from samples of a population extracted in accordance with a second extraction condition so that a ratio of a number of the excluded samples to a number of all samples of the population becomes a given rejection rate.

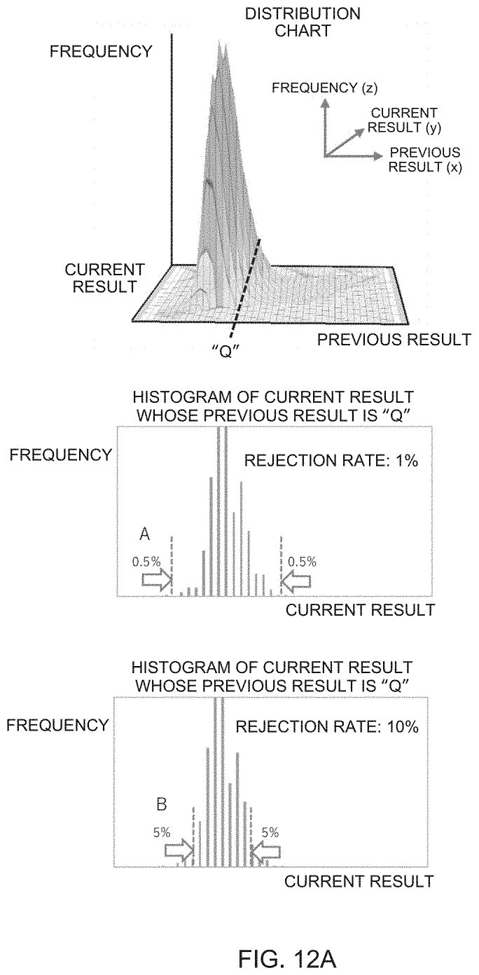

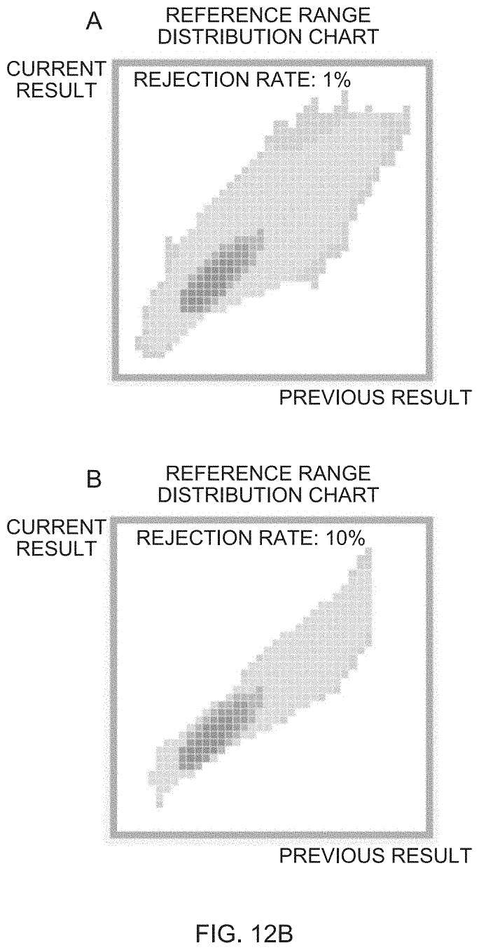

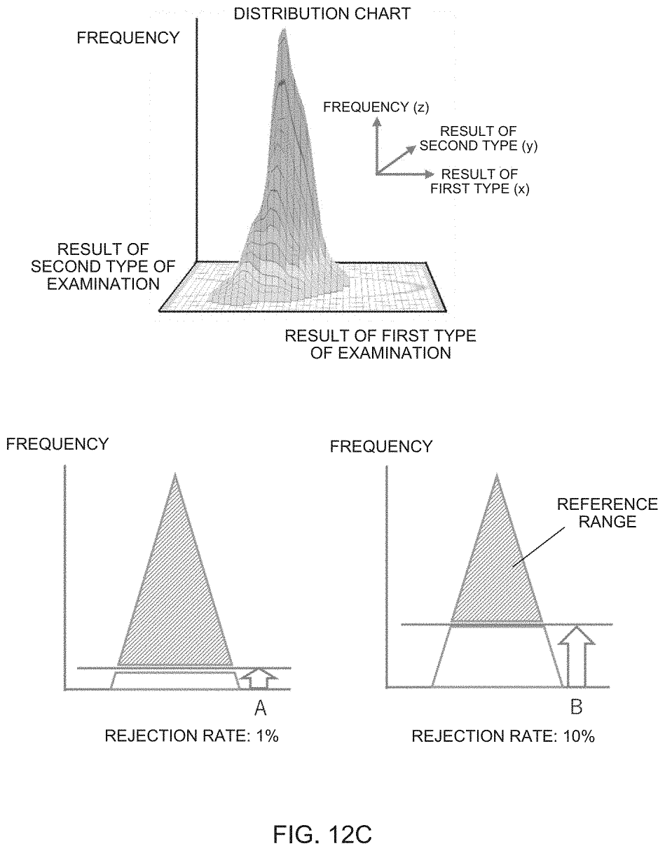

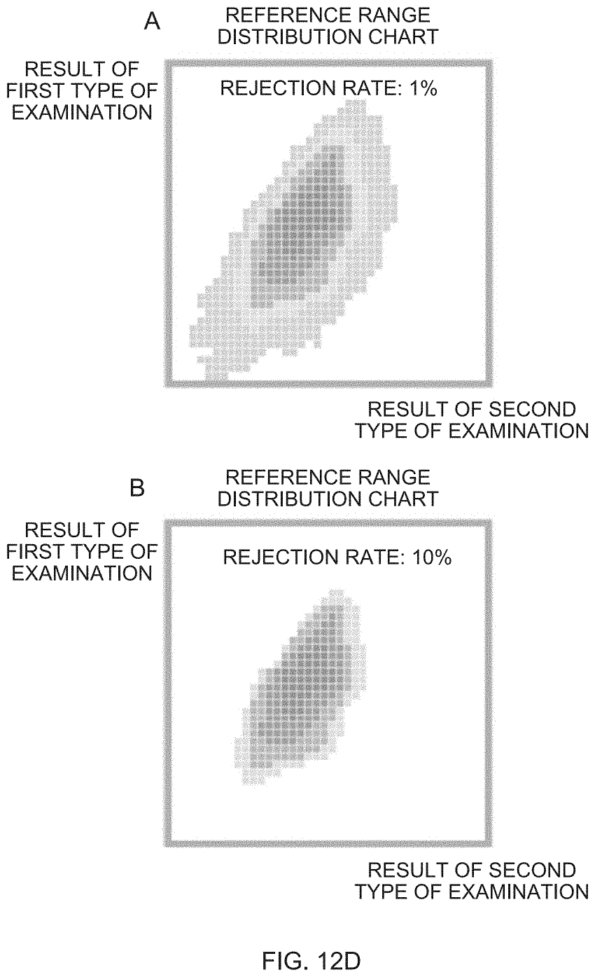

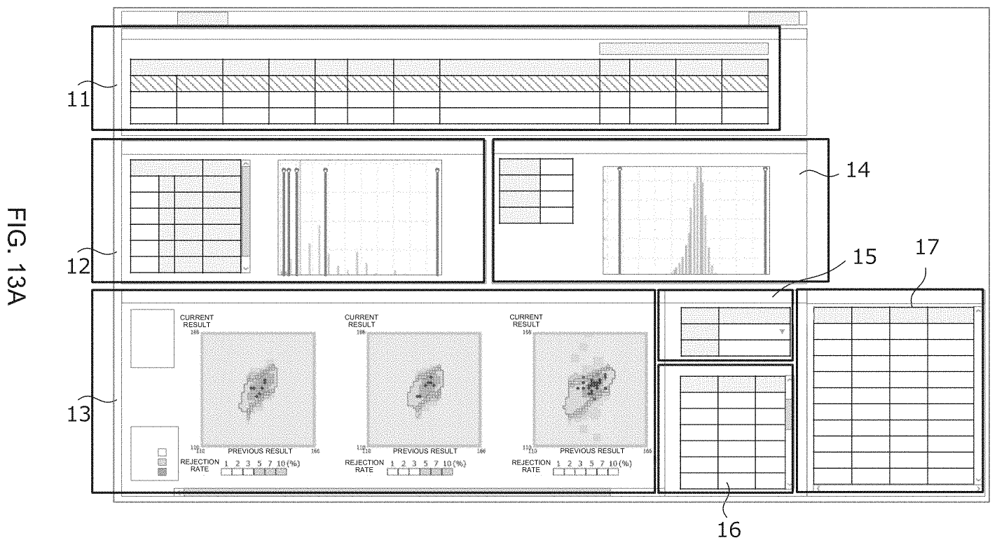

FIGS. 12A and 12B illustrate how sections are determined for exclusion to generate a previous value comparison zone. FIGS. 12C and 12D illustrate how sections are determined for exclusion to generate an inter-item correlation zone. At steps S606 and S607, Distribution Chart Generating Unit 316 generates plural zones by use of different rejection rates so that the operator can compare differences therebetween.

For example, FIGS. 12A and 12B illustrate a previous value comparison zone generated by use of 1% (0.5% from both ends of the x-axis) as the rejection rate, and a previous value comparison zone generated by use of 10% (5% from both ends of the x-axis) as the rejection rate. Similarly, FIGS. 12C and 12D illustrate an inter-item correlation zone generated by use of 1% as the rejection rate, and an inter-item correlation zone generated by use of 10% as the rejection rate.

When Extraction Condition Acquiring Unit 312 acquires a number of days that has elapsed since the previous examination, Distribution Chart Generating Unit 316 counts, for each group of extracted datasets classified by the elapsed number of days, a number of datasets corresponding to each section of a display area, to generate a frequency distribution chart and a reference range distribution chart, i.e. a zone, from the frequency distribution chart, and stores in Work Database 104 a dataset indicating the reference range distribution chart together with a dataset indicating summarized information of the examination results indicated by the datasets used for generating the reference range distribution chart, at steps S604 to S608.

Distribution Chart Generating Unit 316 repeatedly executes the above-mentioned processing to generate a reference range distribution chart for each first extraction condition acquired by Extraction Condition Acquiring Unit 312, at steps S603 to S609. Acquiring Unit 311 acquires a dataset indicating an examination result to be assessed, and Assessing Unit 317 assesses the dataset.

Assessing Unit 317 reads from Work Database 104 a dataset indicating a reference range distribution chart corresponding to a given parameter for assessment, and assesses whether the examination result is normal using the reference range distribution chart indicated by the read dataset. The parameter for assessment is a rejection rate that is used for Distribution Chart Generating Unit 316 to generate a reference range distribution chart. Assessing Unit 317 assesses whether the examination result to be assessed is within a reference range, i.e. a zone, indicated by the reference range distribution chart corresponding to a rejection rate specified by the operator, at steps S701 and S702.

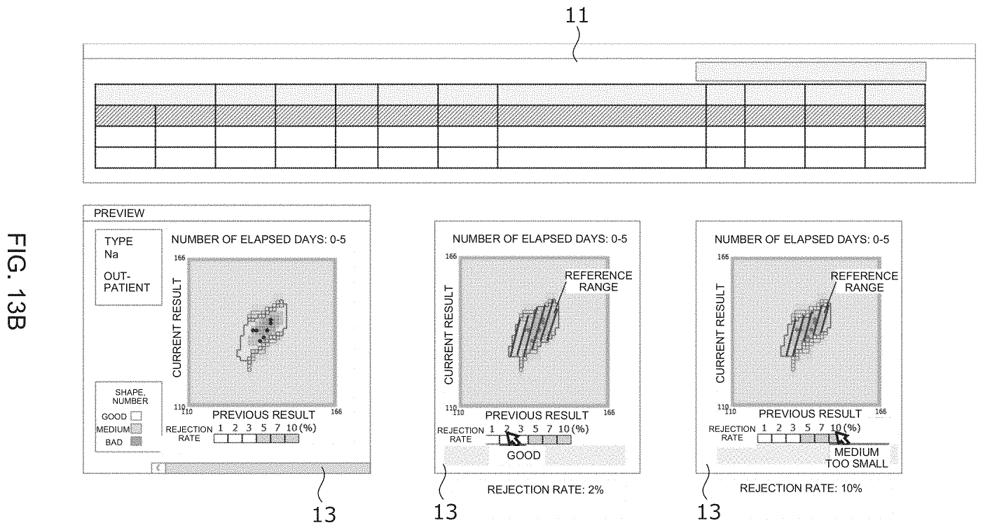

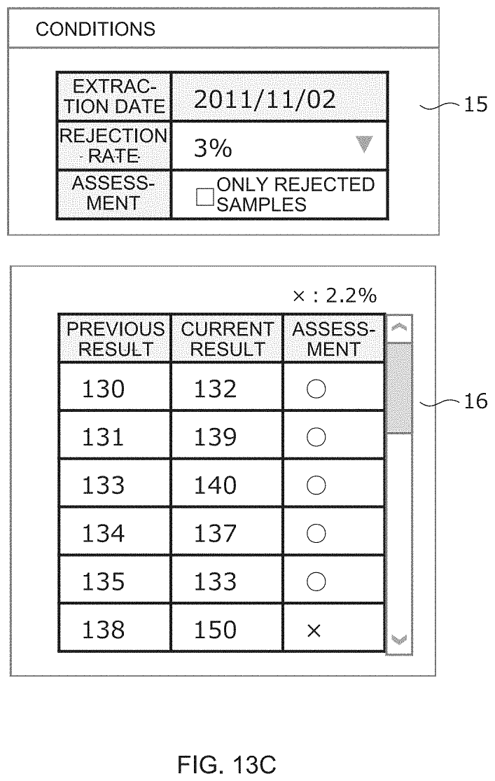

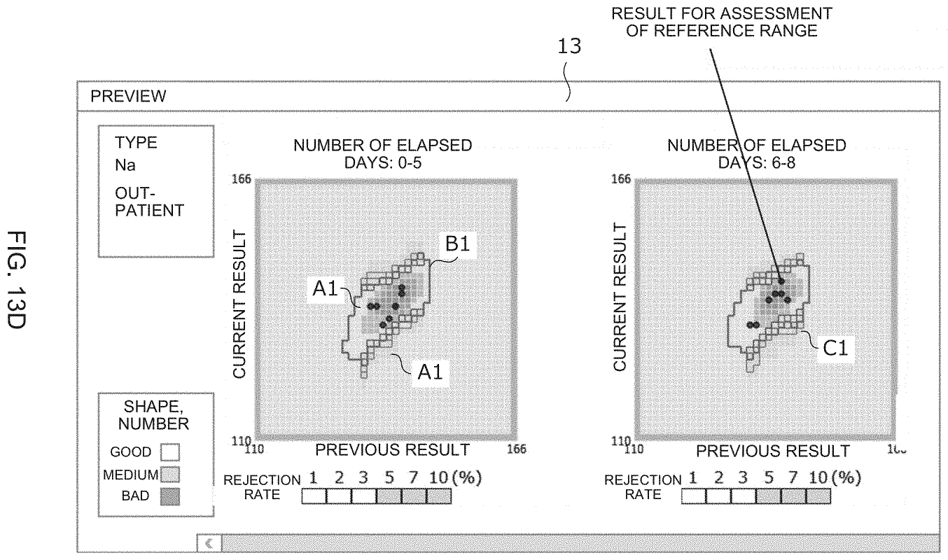

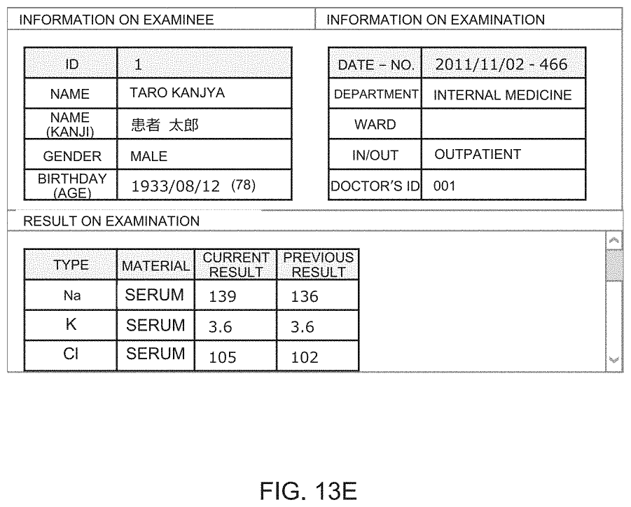

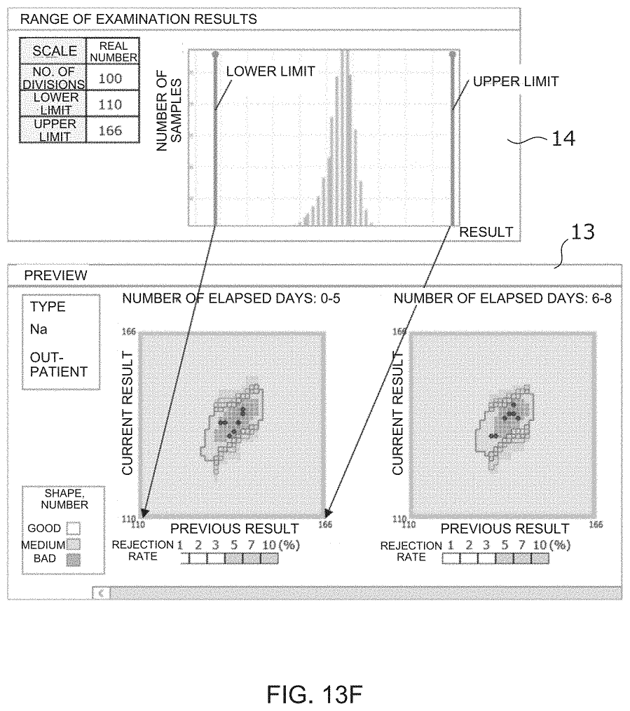

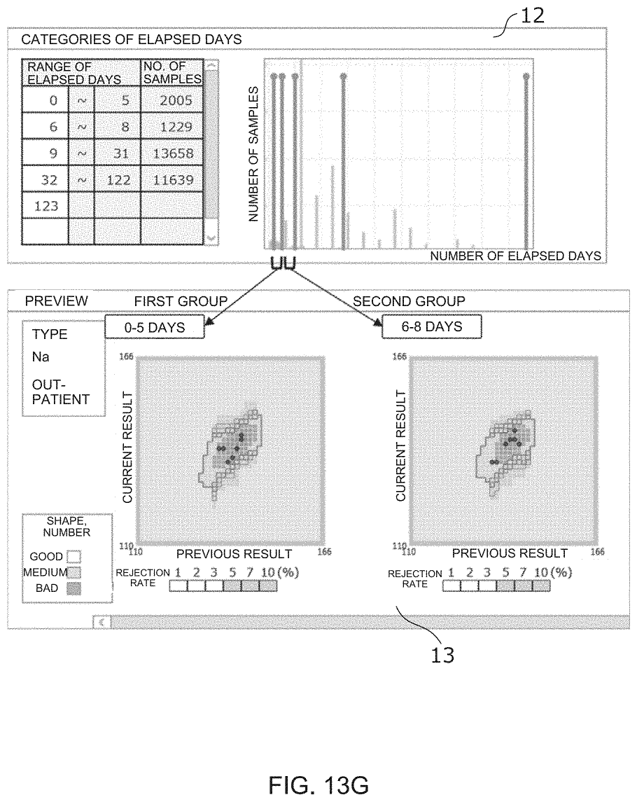

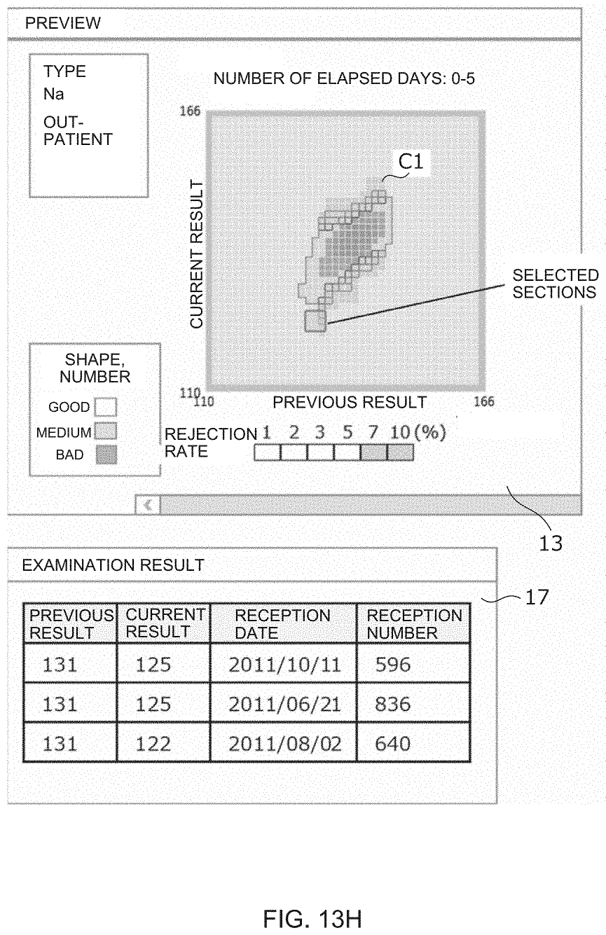

FIG. 13A illustrates an example of a screen displaying information on reference range distribution charts and results of assessment of examination results. Zone Displaying Unit 318 instructs Display 206 of Medical Data Processing Device 102 to display the screen of FIG. 13A including reference range distribution charts generated by Zone Determining Unit 315 and results of assessment performed by Assessing Unit 317 using the reference range distribution charts. In the screen shown in FIG. 13A, previous value comparison zones are displayed.

More specifically, in the screen shown in FIG. 13A, distribution charts with previous value comparison zones relating to sodium concentration in serum are displayed. In Areas 11, 12, 14 and 15 of the screen, default parameters are displayed, and these parameters can be changed by the operator.

In Area 11, conditions for generating distribution charts are displayed. In this case, “sodium” is displayed in Area 11 as one of the conditions. In Area 13, three distribution charts relating to outpatients, inpatients and all patients are displayed. By observing these distribution charts, the operator can obtain information such as, for example, the distribution chart relating to inpatients is unreliable because a number of samples is insufficient. In Areas 12 to 17, information on outpatients and information on inpatients may be displayed in different display styles such as with or without highlighting.

FIG. 13B illustrates Areas 11 and 13 of the screen illustrated in FIG. 13A. FIG. 13C illustrates Areas 15 and 16 of the screen illustrated in FIG. 13A. FIG. 13D illustrates Area 13 of the screen illustrated in FIG. 13A. FIG. 13E illustrates a screen for displaying information on an individual examinee. FIG. 13F illustrates Areas 14 and 13 of the screen illustrated in FIG. 13A. FIG. 13G illustrates Areas 12 and 13 of the screen illustrated in FIG. 13A. FIG. 13H illustrates Areas 13 and 17 of the screen illustrated in FIG. 13A.

As illustrated in FIGS. 13B, 13D and 13F to 13H, the operator can select a rejection rate from among 1%, 2%, 3%, 5%, 7%, and 10%, which are default candidate rejection rates. The default candidate rejection rates may be changed. In Area 12 of the screen, for example, “4” is displayed as a number of categories of elapsed days. According to the number of categories of elapsed days, when the largest number of elapsed days of the samples is, for example, 122, the samples are divided into four groups such as the first group including samples having elapsed days of between 0 and 5, the second group including samples having elapsed days of between 6 and 8, the third group including samples having elapsed days of between 9 and 31, and the fourth group including samples having elapsed days of between 32 and 122. As illustrated in FIG. 13F, the lower limit value, whose default value is the minimum examination result of the samples, and the upper limit value, whose default value is the maximum examination result of the samples, are displayed in a list in Area 14.

In FIGS. 13A to 13H, real numbers are used in the tables or the distribution charts, but logarithmic numbers may be used instead of real numbers if examination results follow a logarithmic distribution. As illustrated in FIG. 13G, a list of sample numbers of each group divided based on the number of elapsed days and a graph indicating a number of samples of each number of elapsed days are displayed in Area 12. As illustrated in FIGS. 13A, 13B, 13D and 13F to 13H, plural distribution charts relating to a sodium concentration for each group of samples divided based on the number of elapsed days are displayed. In FIG. 13A, for example, only three distribution charts for the first, second and third groups are displayed, but the operator can compose a distribution chart for the fourth group, i.e. a distribution chart for samples having elapsed days of between 32 and 122 displayed by dragging a scroll bar.

In the distribution charts in Area 13, a dark portion indicates a high frequency, and the operator can thus readily perceive a frequency of each section by a darkness of the section. For example, as illustrated in FIGS. 13B, 13D and 13F to 13H, outer edges of zones are indicated by borders so that the operator can assess whether the zones are appropriate. FIG. 13F illustrates the relationship between the x-axis of the graph in Area 14 and the x-axis or y-axis of the distribution chart in Area 13. In the graph in Area 14, a number of current examination results that are not excluded for each section is indicated. In the graph in Area 14, the lower limit value and the upper limit value are indicated by vertical solid lines. The range between the minimum examination result and the maximum examination result corresponds to the ranges in the x-axis and y-axis of the distribution chart in Area 13. As illustrated in FIG. 13C, the operator can specify a rejection rate and whether results of assessment shown in Area 16 consist of rejected results only or of all results.

As illustrated in the FIG. 13C, results of assessment of whether each section of the distribution chart is rejected are shown in Area 16. In the list in Area 16, each section is identified by a combination of a previous value and a current value, and a result of assessment of each section following the conditions specified in Area 15 is indicated by circle or an x mark. As illustrated in FIG. 13H, a reception date and a reception number of each of examination results corresponding to one or more sections selected by the operator in the distribution chart in Area 13 is shown in Area 17. A distribution chart to be displayed is changed in accordance with an instruction for changing a parameter received by Distribution Chart Changing Instruction Receiving Unit 319, at step S406.

Display Parameter Changing Instruction Receiving Unit 321 may receive an instruction to change a condition for extracting datasets from Work Database 104. Display Parameter Changing Instruction Receiving Unit 321 receives a new second extraction condition dataset to be replaced with a current second extraction condition dataset. Reading Unit 314 extracts datasets that meet an extraction condition indicated by the new second extraction condition dataset received from Work Database 104.

When datasets are extracted that meet the new second extraction condition, Distribution Chart Generating Unit 316 specifies a new reference range by use of the extracted datasets. Then, Zone Displaying Unit 318 causes Display 206 to display a distribution chart with a new reference range, i.e. a new zone, instead of a distribution chart displayed thus far. Datasets used for generating the new distribution chart are read from Work Database 104, not from Examination Result Database 103. Accordingly, a load on Examination Result Database 103 required for the processing of extracting datasets is reduced.

As already mentioned, FIG. 13B illustrates Areas 11 and 13. In Area 11, the operator can select distribution charts to be displayed in Area 13. In Area 11, a list of parameters of distribution charts generated by Distribution Chart Generating Unit 316 is shown for each of the first extraction conditions. When the operator selects a parameter of distribution charts from the list in Area 11, as illustrated in FIG. 13D, Zone Displaying Unit 318 causes Display 206 to display distribution charts indicated by sign A1 with reference ranges, i.e. zones, indicated by sign B1 determined by use of the selected parameter, i.e. a rejection rate.

Namely, Zone Displaying Unit 318 generates distribution charts indicated by sign A1, which indicate frequencies of samples for each combination of a previous examination result and a current examination result relating to a same examinee, and reference ranges indicated by sign B1, and causes Display 206 to display the distribution charts with the reference ranges on the same coordinate plane.

The operator can change rejection rates by moving a mouse cursor to a square corresponding to a new rejection rate in a bar below the distribution chart in Area 13. Responsive to the mouse cursor operation, Assessment Parameter Changing Instruction Receiving Unit 320 acquires a dataset indicating the new rejection rate to be replaced with a dataset indicating the current rejection rate as a new parameter for assessment at step S407.

Distribution Chart Generating Unit 316 specifies a new reference range in accordance with the new rejection rate indicated by the dataset acquired by Assessment Parameter Changing Instruction Receiving Unit 320. Then, Zone Displaying Unit 318 causes Display 206 to display a distribution chart with the new reference range, i.e. a new zone, instead of a distribution chart with a zone displayed thus far, at step S405. Namely, when the operator moves the mouse cursor to one of the squares in the bar for selecting a rejection rate, Zone Displaying Unit 318 causes Display 206 to display a distribution chart with a zone corresponding to the selected rejection rate.

By performing the above-mentioned operation, the operator can easily follow how the reference range changes as the rejection rate changes, and determine which rejection rate is the best rejection rate for assessing whether each of an examination result is normal. When the operator moves the mouse cursor to one of the squares in the bar for selecting a rejection rate, Zone Displaying Unit 318 also causes Display 206 to display information on the reference range, such as a number of datasets used for generating the reference range, etc.

For example, when a moderate size of distribution chart is displayed around the central area of the display area, assessment information such as “good” may be displayed, and when a small size of distribution chart is displayed in the display area, assessment information such as “too small” may be displayed. Moreover, when a number of datasets used for generating the distribution chart is too small, assessment information such as “insufficient number of samples” may be displayed. Referring to the above-mentioned assessment information, the operator can also easily follow how the reference range changes as the rejection rate changes, and determine which refection rate is the best rejection rate for assessing whether each examination result is normal.

In response to the above-mentioned operation of moving the mouse cursor to one of the squares in the bar for selecting a rejection rate, Assessment Parameter Changing Instruction Receiving Unit 320 receives an instruction to change a parameter for assessment, at step S407.

As already mentioned, FIG. 13C illustrates Areas 15 and 16, and FIG. 13D illustrates Area 13. As illustrated in FIG. 13C, Area 15 is an area for the operator to input a parameter for assessment, i.e. a rejection rate, and Area 16 is an area for displaying results of assessment of whether each section of the distribution chart is rejected in accordance with the rejection rate specified in Area 15. As illustrated in FIG. 13D, Area 13 is an area for displaying distribution charts. When the operator changes rejection rates in Area 15, for each first extraction condition, Acquiring Unit 311 may acquire datasets for assessing a rejection rate, and Assessing Unit 317 assesses examination results indicated by the datasets for assessing a rejection rate by use of the rejection rate specified by the operator.

The datasets for assessing a rejection rate are not the datasets extracted from Work Database 104 by use of the second extraction condition to be evaluated, but are datasets prepared in advance for assessing a rejection rate specified by the operator. The datasets for assessing a rejection rate are examples of “datasets for assessing” described in the claims of the present application. Acquiring Unit 311 that acquires datasets for assessing a rejection rate is an example of a “sixth acquiring unit” described in the claims of the present application. Each of datasets for assessing a rejection rate indicates an examination result, as well as an item of examination, a date of examination, an identifier of an examinee, one or more attributes of an examinee, etc., similarly to datasets for assessment.

In an exemplary embodiment, Acquiring Unit 311 extracts datasets for assessing a rejection rate from Work Database 104 by use of an extraction condition that is different from a second extraction condition. Datasets for assessing a rejection rate may be extracted from Examination Result Database 103 instead of Work Database 104. However, when datasets for assessing a rejection rate are extracted from Work Database 104, a load on Examination Result Database 103 is reduced.

Assessing Unit 317 assesses whether each examination result acquired by Acquiring Unit 311 falls within the reference range when the rejection rate specified by the operator is applied to the selected distribution chart. Zone Displaying Unit 318 causes Display 206 to display a list of results of the assessment for examination results indicated by the datasets for assessing a rejection rate and a ratio of a number of rejected sections to a number of total sections in Area 16. Zone Displaying Unit 318 also causes Display 206 to display plots indicating examination results for assessing a rejection rate in the distribution chart in Area 13.

Namely, as illustrated in FIG. 13D, Zone Displaying Unit 318 causes Display 206 to display, in the display area in Area 13, a graph including the distribution chart and plots placed on sections of the distribution chart corresponding to examination results indicated by the datasets for assessing a rejection rate.

By referring to the above-mentioned plots on the distribution chart, the operator can easily follow how the results of assessing whether examination results fall within the reference range changes as the rejection rate changes, and determine which refection rate is the best rejection rate for assessing whether an examination result is normal. In this exemplary embodiment, the operator can also change the parameter for assessment, i.e. the rejection rate, by carrying out a simple operation via the screen. When the rejection rate is changed by the operator, the distribution chart and the list of results of assessment are updated without extracting datasets from Examination Result Database 103, whose load is greater than that of Work Database 104. Accordingly, the operator can efficiently determine the best rejection rate.

Zone Displaying Unit 318 causes Display 206 to display the distribution chart so that the operator can select any of the plots on the distribution chart. Namely, the distribution chart with the plots in Area 13 functions as a screen of a user interface that receives a selection of sections where one or more examination results for assessing a rejection rate is made by the operator. The screen of the user interface is an example of a “fourth interface screen” described in the claims of the present application.

As already mentioned, FIG. 13E illustrates a screen for displaying information on an individual examinee. The information shown in FIG. 13E is, for example, information shown when one of the plots on the distribution chart is selected by the operator. Zone Displaying Unit 318 causes Display 206 to display information on one or more examinees whose samples correspond to a section that is selected by the operator on the screen that functions as a user interface. When there are plural samples that correspond to one or more sections selected by the operator, all information on the samples may not be displayed on the screen at the same time.

When all of the information on the samples selected by the operator cannot be displayed at the same time, Zone Displaying Unit 318 may cause Display 206 to display only part of the information such as a reception number for each sample at the same time. In this case, display of a list of information such as a reception number of each sample may function as a user interface, and Zone Displaying Unit 318 may cause Display 206 to display all information on a sample that is selected by the operator when the operator carries out an operation on the list to select the sample. In this case, the operator can also refer to detailed information on samples for assessing a rejection rate when the operator determines the best rejection rate.

Display Parameter Changing Instruction Receiving Unit 321 receives an instruction to change parameters for displaying a distribution chart, at step S408.

As already mentioned, FIG. 13F illustrates Areas 14 and 13. In Area 14, the operator can change the lower limit value and the upper limit value. In Area 13, distribution charts are displayed.

When a reference range, i.e. a zone, is located in the central portion of the display area without contacting the outer frame of the display area and its size is not too small, the operator can easily understand the distribution of normal and abnormal values. Accordingly, the operator can adjust the range of the display area so that the reference range is displayed in a moderate size in the central portion of the display area.

Zone Displaying Unit 318 causes Display 206 to display in Area 14 a histogram with an x-axis representing an examination result and y-axis representing a frequency generated by use of the same datasets used to generate a selected distribution chart in Area 13. The vertical solid lines in the histogram indicate the lower limit value and the upper limit value, which indicate the range of the x-axis and y-axis of the display area of the selected distribution chart in Area 13.

The operator can move each of the vertical solid lines in the histogram in the x-axis direction by use of Input Device 208 such as a mouse and touch panel. Zone Displaying Unit 318 acquires a dataset indicating a new lower limit value or a new upper limit value in response to the operation made by the operator to move one of the solid lines. The dataset indicating a new lower limit value or a new upper limit value also indicates a new low end value or a new high end value of the x-axis and y-axis of the display area of the selected distribution chart in Area 13. The dataset indicating a lower limit value or an upper limit value is an example of a “dataset indicating a range of coordinate axes” described in the claims of the present application. Zone Displaying Unit 318 acquiring a dataset indicating a lower limit value or a upper limit value is an example of a “fifth acquiring unit” described in the claims of the present application.

When Zone Displaying Unit 318 acquires a dataset indicating a new lower limit value or a new upper limit value, Zone Displaying Unit 318 causes Display 206 to display a new distribution chart with a new display area whose range in the x-axis or y-axis is changed by the new lower limit value or the new upper limit value indicated by the acquired dataset. In FIG. 13F, the display area of the distribution chart in the left side of Area 13 has an x-axis and a y-axis whose ranges are between the upper limit value and the lower limit value indicated by the vertical solid lines in the histogram in Area 14. The histogram indicates a number of samples of each examination-result value. The samples used for generating the histogram are datasets extracted from Work Database 104 by use of a second extraction condition.

As illustrated in FIG. 13A, Zone Displaying Unit 318 causes Display 206 to display a screen that includes a histogram and receives an operation carried out by the operator on the histogram. The screen illustrated in FIG. 13A is an example of a “second interface screen” described in the claims of the present application.

Zone Displaying Unit 318 acquires a dataset indicating a lower limit value or an upper limit value that is input by the operator via the screen. When the operator inputs a new lower limit value or a new upper limit value, Zone Displaying Unit 318 acquires a dataset indicating the new lower limit value or the new upper limit value.

When Zone Displaying Unit 318 acquires a dataset indicating a new lower limit value or a new upper limit value, Zone Displaying Unit 318 causes Display 206 to display a new distribution chart with a display area with ranges in the x-axis direction and y-axis direction whose low end is the new lower limit value or whose high end is the new upper limit value, instead of the distribution chart displayed thus far. Zone Displaying Unit 318 causes Display 206 to display a new distribution chart immediately after Zone Displaying Unit 318 acquires a new lower limit value or a new upper limit value.

When a new distribution chart is displayed, Assessing Unit 317 assesses each examination result by use of the new distribution chart.

By referring to the histogram, the operator can easily adjust the range of display area of the distribution chart while assessing a distribution of examination results. The x-axis of a histogram in Area 14, the x-axis of a distribution chart in Area 13, and the y-axis of a distribution chart in Area 13 may be a real number scale or a logarithmic scale. When the scale of the x-axis of histogram in Area 14 is changed by the operator, Zone Displaying Unit 318 generates a new histogram with the x-axis of the newly specified scale and a new distribution chart with the x-axis and y-axis of the newly specified scale, and causes Display 206 to display the new histogram and the new distribution chart.

When a new distribution chart is displayed, Assessing Unit 317 assesses each examination result by use of the new distribution chart.

As described above, according to this exemplary embodiment, the operator can easily change parameters for displaying a distribution chart such as a range of a display area of the distribution chart, a type of axes, etc. by carrying out a simple operation via the screen, and confirm changes in appearance of a reference range in response to the change of parameters.

As already mentioned, FIG. 13G illustrates Areas 12 and 13. In Area 12, the operator can specify a range of a number of elapsed days for each group of datasets. In Area 13, distribution charts corresponding to the groups of datasets are displayed. A number of days that has elapsed between the previous examination and the current examination for the same examinee is a parameter that significantly affects a difference between results of the previous examination and the current examination. Accordingly, by classifying examination results into plural groups according to the numbers of days that has elapsed between the previous examination and the current examination for the same examinee, it is possible to accurately assesses whether an examination result is normal by use of a reference range.

Zone Displaying Unit 318 causes Display 206 to display in Area 12, a histogram with an x-axis representing a number of days that has elapsed from a previous examination and a y-axis representing a frequency of examination results. The histogram in Area 12 is generated using the same datasets used to generate distribution charts in Area 13. Vertical solid lines other than bars indicating frequencies in the histogram indicate boundary values of a number of elapsed days for use in classifying datasets into the groups.

The operator can move any of the vertical solid lines by using Input Device 208 such as a mouse and touch panel in the x-axis direction. When one of the vertical solid lines is moved by the operator, Zone Displaying Unit 318 acquires a dataset indicating new ranges of a number of elapsed days for classifying the datasets into plural groups.

The dataset indicating ranges of a number of elapsed days for classifying the datasets into plural groups is an example of a “dataset indicating a boundary value of a number of elapsed days” described in the claims of the present application. Zone Displaying Unit 318 for acquiring a dataset indicating ranges of a number of elapsed days for classifying the datasets into plural groups is an example of a “fourth acquiring unit” described in the claims of the present application. When Zone Displaying Unit 318 acquires a dataset indicating new ranges of a number of elapsed days, Distribution Chart Generating Unit 316 classifies the datasets indicating examination results to be assessed into new groups, whose ranges of numbers of elapsed days are indicated by the dataset acquired by Zone Displaying Unit 318, generates a new distribution chart for each of the new groups, and specifies a new reference range for each of the new groups.

Zone Displaying Unit 318 causes Display 206 to display the new distribution chart with the new reference range for each of the new groups in Area 13. As described above, Zone Displaying Unit 318 causes Display 206 to display a screen including a histogram indicating frequencies of examination results for each number of days that has elapsed between the previous examination and the current examination for the same examinee, and the screen accepts an operation carried out by the operator. The screen is an example of a “first interface screen” described in the claims of the present application.

Zone Displaying Unit 318 acquires a dataset indicating new boundary values for classifying datasets into plural groups responsive to an operation carried out via the screen by the operator. When Zone Displaying Unit 318 acquires a new dataset indicating boundary values for classifying datasets into plural groups, Zone Displaying Unit 318 replaces the new dataset with the old dataset

When Zone Displaying Unit 318 acquires a dataset indicating new boundary values of a number of elapsed days, Distribution Chart Generating Unit 316 classifies the datasets indicating examination results to be assessed into new groups, whose ranges of a number of elapsed days are indicated by the dataset acquired by Zone Displaying Unit 318, generates a new distribution chart for each of the new groups, and specifies a new reference range for each of the new groups. Zone Displaying Unit 318 causes Display 206 to display the new distribution charts with the new reference ranges instead of the distribution charts displayed thus far. Zone Displaying Unit 318 causes Display 206 to display the new distribution charts in Area 13 immediately after the operator carries out an operation of moving one of the vertical solid lines in the histogram in Area 12.

When new distribution charts are displayed, Assessing Unit 317 assesses each examination result by use of the new distribution charts. By referring to the histogram in Area 12, the operator can easily adjust the ranges of groups of datasets to be assessed while assessing a distribution of examination results for each number of elapsed days.

As described above, according to this exemplary embodiment, the operator can easily change ranges of a number of elapsed days for classifying examination results into plural groups by carrying out a simple operation via the screen, and confirm changes of reference ranges responsive to the change of ranges of the number of elapsed days.

As already mentioned, FIG. 13H illustrates Areas 13 and 17. In Area 17, examination results used for generating a selected distribution chart in Area 13 are displayed. The operator can select one or more sections of the distribution chart in Area 13.

Namely, Zone Displaying Unit 318 causes Display 206 to display distribution charts, which are graphs for indicating a distribution of examination results indicated by datasets extracted from Work Database 104 using a second extraction condition, as a user interface that receives an operation made by the operator. The screen including the distribution charts for receiving an operation carried out by the operator is an example of a “third interface screen” described in the claims of the present application.

In FIG. 13H, a group of sections with sign C2 are selected by the operator. Zone Displaying Unit 318 causes Display 206 to display at least part of information on examination results corresponding to the selected sections in Area 17. In FIG. 13H, as an example, information on three examination results, that may be either examination results for different examinees or for the same examinee, is displayed in Area 17.

When any of the rows in the list in Area 17 is selected by the operator, Zone Displaying Unit 318 causes Display 206 to display information on an examinee whose examination result is selected as illustrated in FIG. 13E. Referring to the information on the examinee, the operator can consider, for example, a reason why an usual shape occurs in a reference range such as a shape including one or more small detached portions, etc.

When one or more sections in a distribution chart is selected by the operator, Zone Displaying Unit 318 acquires datasets corresponding to the selected sections from Work Database 104, and causes Display 206 to display information indicated by the datasets in Area 17. When one of the rows in the list in Area 17 is selected by the operator, Zone Displaying Unit 318 causes Display 206 to display detailed information on an examinee whose examination result is selected. Referring to the detailed information as illustrated in FIG. 13E, the operator can efficiently determine the best reference range.

After assessing parameters for displaying distribution charts and for evaluating examination results, the operator selects a rejection rate, and Reference Range Selecting Instruction Receiving Unit 322 receives a dataset indicating the selected rejection rate. Reference Range Determining Unit 323 determines, for each extraction condition, a reference range used for assessing whether an examination result is normal.

When Reference Range Selecting Instruction Receiving Unit 322 receives a dataset indicating a rejection rate selected by the operator for each extraction condition at step S409, Reading Unit 324 reads datasets from Work Database 104 in accordance with parameters acquired by Zone Displaying Unit 318. Reference Range Determining Unit 323 determines a reference range for each extraction condition using the datasets read by Reading Unit 324.

As described above, in the present exemplary embodiment, reference ranges are efficiently determined by use of datasets read from Work Database 104 without placing a load on Examination Result Database 103, and a burden on the operator is also reduced since a time required for extracting datasets is shortened. When Medical Data Processing Device 102 acquires examination results from the analyzer, Examination Result Assessing Unit 302 selects from reference ranges determined by Reference Range Determining Unit 323, for each of the examination results, a reference range for the examination result, and assesses whether the examination result is normal by use of the selected reference range.

Display 206 displays a screen including results of the assessments performed by Examination Result Assessing Unit 302.

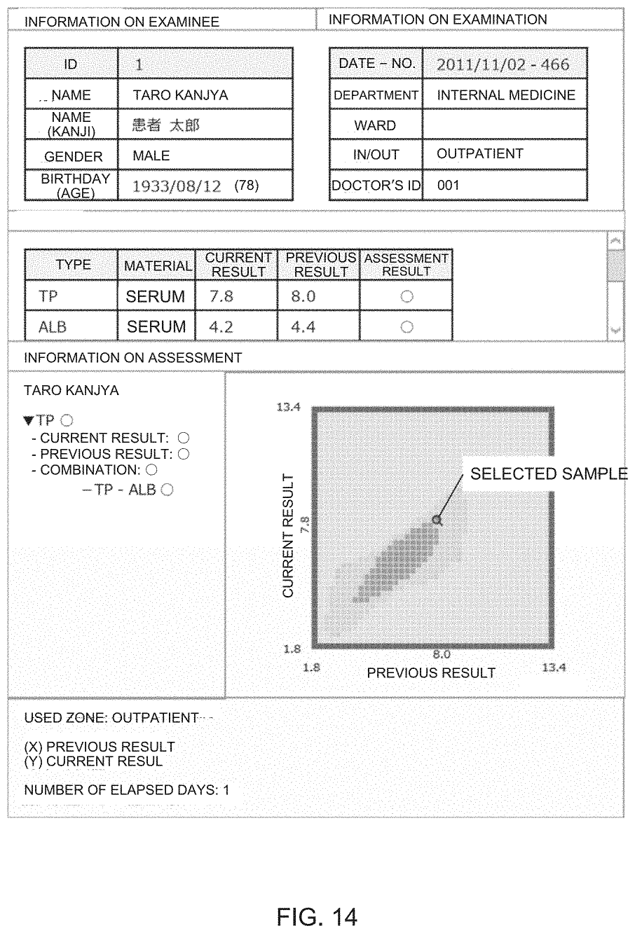

FIG. 14 illustrates a screen including results of the assessments. On the screen, results of assessments performed by Examination Result Assessing Unit 302 show whether each examination result for the examinee is normal using the selected reference range, i.e. the selected zone.

As already mentioned, several different types of distribution charts, such as a distribution chart indicating a relationship between results of a previous examination and a current examination for the same examinee, a distribution chart indicating a relationship between results of two different types of examinations performed at the same time for the same examinee, etc., may be generated by Medical Data Processing Device 102. The operator selects a type of distribution chart such that a zone of the selected type of distribution chart is suitable for use in assessing examination results considering a nature of examination whose results are assessed, etc. In the screen illustrated by FIG. 14, Display 206 displays a result of assessment of whether an examination result is normal, as well as a distribution chart used for the assessment with a plot indicating a section corresponding to the examination result and a type of the distribution chart.

Examination Result Assessing Unit 302 assesses an examination result within the reference range as normal, and assesses an examination result outside the reference range as abnormal. When Medical Data Processing Device 102 acquires examination results from the analyzer, Display 206 can immediately display an assessment result showing whether each of the examination results is normal or abnormal. Accordingly, when an abnormal examination result is found, the operator can promptly inform the result to a laboratory technician who performed the examination so that the laboratory technician can prevent occurrence of similar abnormal examination results.

It is necessary to assess whether a result of medical examination is normal or abnormal, and when the result is abnormal, the medical examination must be redone. To assess whether a result of medical examination is normal or abnormal, it is necessary to determine an appropriate reference range for each item of examination taking into consideration several conditions of the examination such as a gender of an examinee, a department where the examinee was seen by a doctor, whether the examinee is an outpatient or inpatient, etc.

Accordingly, it is necessary for the operator to repeatedly generate a reference range by changing parameters and determine whether the reference range is appropriate for assessing the given examination results until a suitable reference range is arrived at. According to this embodiment, it is possible for Medical Data Processing Device 102 to perform processing to efficiently find a suitable reference range by temporarily storing in Work Database 104 datasets extracted from Examination Result Database 103, and generating a distribution chart with a reference range using the datasets stored in Work Database 104.

Work Database 104 may be configured as a part of Medical Data Processing Device 102, instead of being configured as a separate device from Medical Data Processing Device 102. In this case, the above-mentioned processing for finding a suitable reference range can also be performed efficiently in that Work Database 104 is separate from Examination Result Database 103. As a result, according to Medical Data Processing Device 102, the operator can efficiently assess whether each examination result is normal without being subjected to an excessive workload, a load on Medical Data Processing Device 102 is minimized, and stable operation of Medical Data Processing System 101 is ensured.

### [2] Modifications

The above-described embodiment is an exemplary embodiment of the present invention, and may be modified in various ways. Following are examples of modifications of the above-described embodiment. Two or more of the above-mentioned embodiment and the following modifications may be combined.

### [2-1] Viewpoint for Assessing Examination Result

In the above-described embodiment, an examination result is assessed from a viewpoint of comparison with a previous examination result for the same examinee. An examination result may be assessed from a viewpoint other than the viewpoint of comparison with a previous examination result for the same examinee. For example, an examination result may be assessed from a viewpoint of comparison with a result of a different type of examination performed at the same time for the same examinee. For example, a result of examination on GOT (Glutamic Oxaloacetic Transaminase) is assessed based on relationship between the result of examination on GOT and a result of examination on GPT (Glutamic Pyruvic Transaminase) performed at the same time for the same examinee.

Other combinations of two items of examination may be selected for assessing one of the items. For example, any two of sodium concentration, blood glucose level, total protein, and lactate DeHydorogenase may be selected. Namely, a first result of examination may be assessed in comparison with a second result of examination performed for the same examinee under different conditions.

### [2-2] Configuration of Devices for Realizing Functions of the System

As far as the functional configuration illustrated in FIG. 3 is realized, the configuration of Medical Data Processing System 101 is not limited to the configuration illustrated in FIG. 1. For example, one or more functions realized by Medical Data Processing Device 102 in the above-described embodiment may be realized by a device other than Medical Data Processing Device 102, or by Medical Data Processing Device 102 and one or more other data processing devices in cooperation.

### [2-3] Category of Invention

The present invention may be understood as a data processing device exemplified by Medical Data Processing Device 102 and a data processing system including the data processing device exemplified by Medical Data Processing System 101. The data processing system may consist of a group of plural distributed computer resources that operate in cooperation, such as a cloud computing system. When Medical Data Processing Device 102 is realized by such a group of plural distributed computer resources, Medical Data Processing Device 102 is an example of a “system” described in the claims of the present application as well as Medical Data Processing System 101.

The present invention may also be understood as a data processing method consisting of steps executed by the above-mentioned data processing device or the above-mentioned data processing system, and as a program for causing a computer to execute the steps of the data processing method. The program may be provided in a form of a recording medium such as an optical disk on which the program is stored, or may be provided in a form of downloading to a computer via a network such as the Internet and installed in the computer.

### INDUSTRIAL APPLICABILITY

A data processing device, a data processing system, a program, and a data processing method according to the present invention are useful, for example, for assessing a validity of a result of medical examination taking into consideration various information such as a history of treatments made to the examinee, a change in results of the same medical examination over time, etc. Accordingly, the present invention may be implemented in a medical institution or the like where a rapid and efficient assessment of medical examination results is required.

### DESCRIPTION OF REFERENCE NUMERALS

101: Medical Data Processing System
102: Medical Data Processing Device
103: Examination Result Database
104: Work Database
301: Zone Generating Unit
302: Examination Result Assessing Unit
311: Acquiring Unit
312: Extraction Condition Acquiring Unit
313: Examination Result Extracting Unit
314: Reading Unit
315: Zone Determining Unit
316: Distribution Chart Generating Unit
317: Assessing Unit
318: Zone Displaying Unit
319: Distribution Chart Changing Instruction Receiving Unit
320: Assessment Parameter Changing Instruction Receiving Unit
321: Display Parameter Changing Instruction Receiving Unit
322: Reference Range Selecting Instruction Receiving Unit
323: Reference Range Determining Unit
324: Reading Unit

1. A system comprising:
a first acquiring unit that acquires a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee,
a generating unit that extracts from the first database datasets that satisfy the first extraction condition, and generates a second database that stores the datasets extracted from the first database,
a storage unit that stores the second database,
a second acquiring unit that acquires a second extraction condition dataset indicating a second extraction condition for extracting datasets from the second database, the second extraction condition relating to at least one of an item of extraction, a period of examination, and an attribute of an examinee,
a third acquiring unit that acquires a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions,
an extraction unit that extracts from the second database datasets that satisfy the second extraction condition,
a specifying unit that specifies a reference range within which samples that are not rejected from a population in accordance with the rejection rate are distributed, the population being generated based on the datasets extracted from the second database and consisting of samples, each of which indicates a result of the first examination and a result of the second examination, and
a display unit that displays a graph with a first coordinate axis representing a result of the first examination and a second coordinate axis representing a result of the second examination, the graph indicating the reference range.

2. A system according to claim 1, wherein:
when the second acquiring unit acquires a new second extraction condition dataset to be replaced with the second extraction condition dataset already acquired by the second acquiring unit,
the extraction unit extracts from the second database datasets that satisfy a new second extraction condition indicated by the new second extraction condition dataset,
the specifying unit specifies a new reference range by use of the datasets extracted from the second database by use of the new second extraction condition, and
the display unit displays a new graph indicating the new reference range instead of the graph displayed thus far.

3. A system according to claim 1, wherein:
when the third acquiring unit acquires a new rejection rate dataset to be replaced with the rejection rate dataset already acquired by the third acquiring unit,
the specifying unit specifies a new reference range in accordance with a new rejection rate indicated by the new rejection rate dataset, and
the display unit displays a new graph indicating the new reference range instead of the graph displayed thus far.

4. A system according to claim 1, wherein:
when the first examination is performed at a first timing and the second examination is performed at a second timing later than the first timing,
the system further comprises a fourth acquiring unit that acquires a boundary value dataset indicating one or more boundary values of a number of days elapsed between the first timing and the second timing, the one or more boundary values being used for classifying datasets into groups,
the specifying unit specifies a reference range for each of groups of the datasets extracted from the second database classified by use of the one or more boundary values, and
the display unit displays graphs indicating the reference ranges specified by the specifying unit for each of the groups.

5. A system according to claim 4, wherein:
when the fourth acquiring unit acquires a new boundary value dataset to be replaced with the boundary value dataset already acquired by the fourth acquiring unit,
the specifying unit specifies a new reference range for each of new groups of the datasets extracted from the second database classified by use of one or more boundary values indicated by the new boundary value dataset, and
the display unit displays graphs indicating the new reference ranges specified by the specifying unit for each of the new groups.

6. A system according to claim 4, wherein:
the display unit displays a first interface screen that includes a graph indicating a number of samples in the population of each number of days elapsed between the first timing and the second timing, the first interface screen receiving an operation carried out by a user on the graph, and
the fourth acquiring unit acquires a boundary value dataset indicating one or more boundary values that are input by the user via the first interface screen.

7. A system according to claim 1, wherein:
the system further comprises a fifth acquiring unit that acquires an axis range dataset indicating displayed ranges of a first coordinate axis and a second coordinate axis of a graph indicating a reference range,
the display unit displays a graph indicating the reference range specified by the specifying unit, the graph having a first coordinate axis and a second coordinate axis whose displayed ranges are indicated by the axis range dataset, and
when the fifth acquiring unit acquires a new axis range dataset to be replaced with the axis range dataset already acquired by the fifth acquiring unit, the display unit displays a new graph indicating the reference range with the first coordinate axis and the second coordinate axis whose displayed ranges are indicated by the new axis range dataset instead of the graph displayed thus far.

8. A system according to claim 7, wherein:
the display unit displays a second interface screen that includes a graph indicating a number of samples in a population of each examination-result value, the population being generated based on the datasets extracted from the second database, the second interface screen receiving an operation carried out by a user on the graph, and
the fifth acquiring unit acquires an axis range dataset indicating a range of the first coordinate axis and the second coordinate axis that is input by the user via the second interface screen.

9. A system according to claim 1, wherein:
the display unit displays a graph including a distribution chart indicating a distribution of samples in a population and the reference range on a same coordinate plane, the population being generated based on the datasets extracted from the second database and consisting of samples each of which indicates a result of the first examination and a result of the second examination.

10. A system according to claim 9, wherein:
the display unit displays a third interface screen that includes the graph including the distribution chart and the reference range, the third interface screen receiving an operation carried out by a user on the graph, and
the display unit displays information on a sample specified by the user via the third interface screen.

11. A system according to claim 1, wherein:
the system further comprises a sixth acquiring unit that acquires datasets for assessment, the datasets for assessment relating to clinical examinations, each of the datasets for assessment indicating an item of examination, an examination-result value, a date of examination, an identifier of an examinee, and an attribute of the examinee, the datasets for assessment being different from the datasets extracted from the second database by use of the second extraction condition, and
the display unit displays a graph including a distribution chart indicating distribution of samples in a population and the reference range on a same coordinate plane, the population being generated based on the datasets for assessment and consisting of samples each of which indicates a result of the first examination and a result of the second examination.

12. A system according to claim 11, wherein:
the extraction unit extracts from the second database the datasets for assessment by use of an extraction condition that is different from the second extraction condition.

13. A system according to claim 11, wherein:
the display unit displays a fourth interface screen that includes a graph including a distribution chart indicating a distribution of samples in a population and the reference range on a same coordinate plane, the population being generated based on the datasets for assessment, the fourth interface screen receiving an operation carried out by a user on the graph, and
the display unit displays information on a sample specified by the user via the fourth interface screen.

14. A program causing a computer to execute:
a first acquiring step for acquiring a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee,
a generating step for extracting from the first database datasets that satisfy the first extraction condition, and generating a second database that stores the datasets extracted from the first database,
a storing step for storing the second database,
a second acquiring step for acquiring a second extraction condition dataset indicating a second extraction condition for extracting datasets from the second database, the second extraction condition relating to at least one of an item of extraction, a period of examination, and an attribute of an examinee,
a third acquiring step for acquiring a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions,
an extraction step for extracting from the second database datasets that satisfy the second extraction condition,
a specifying step for specifying a reference range within which samples that are not rejected from a population in accordance with the rejection rate are distributed, the population being generated based on the datasets extracted from the second database and consisting of samples, each of which indicates a result of the first examination and a result of the second examination, and
a displaying step for displaying a graph with a first coordinate axis representing a result of the first examination and a second coordinate axis representing a result of the second examination, the graph indicating the reference range.

15. A method executed by a data processing device comprising:
a first acquiring step for acquiring a first extraction condition dataset indicating a first extraction condition for extracting datasets from a first database, the first database storing datasets relating to clinical examinations, each of the datasets stored in the first database indicating an item of examination, an examination-result value, a date of examination, an identifier of an examinee, and an attribute of the examinee, the first extraction condition relating to at least one of an item of extraction, a period of examination, and an attribute of an examinee,
a generating step for extracting from the first database datasets that satisfy the first extraction condition, and generating a second database that stores the datasets extracted from the first database,
a storing step for storing the second database,
a second acquiring step for acquiring a second extraction condition dataset indicating a second extraction condition for extracting datasets from the second database, the second extraction condition relating to at least one of an item of extraction, a period of examination, and an attribute of an examinee,
a third acquiring step for acquiring a rejection rate dataset indicating a rejection rate which is a ratio of a number of samples to be rejected from a population to a number of total samples in the population, each of the samples in the population indicating a result of first examination and a result of second examination, the first examination and the second examination being performed for a same examinee under different conditions,
an extraction step for extracting from the second database datasets that satisfy the second extraction condition,
a specifying step for specifying a reference range within which samples that are not rejected from a population in accordance with the rejection rate are distributed, the population being generated based on the datasets extracted from the second database and consisting of samples, each of which indicates a result of the first examination and a result of the second examination, and
a displaying step for displaying a graph with a first coordinate axis representing a result of the first examination and a second coordinate axis representing a result of the second examination, the graph indicating the reference range.
